(19) United States
(12) Patent Application Publication
(10) Pub. No.: US 2025/0009235 A1
(43) Pub. Date: Jan. 9, 2025

(54) CONTACTLESS THREE DIMENSIONAL ELECTRO-MAGNETIC ULTRASOUND SYSTEM

(71) Applicant: Emanuele Raggi, PROVIDENCE, RI (US)

(72) Inventor: Emanuele Raggi, PROVIDENCE, RI (US)

(21) Appl. No.: 18/190,099

(22) Filed: Mar. 26, 2023

Related U.S. Application Data
(60) Provisional application No. 63/303,410, filed on Jan. 26, 2022.

(51) Int. Cl. A61B 5/00; A61B 5/06; G01S 15/89; A61B 90/00

(52) U.S. Cl. CPC A61B 5/0095; A61B 5/067; A61B 5/6887; A61B 5/725; G01S 15/897; G01S 15/899; G01S 15/8993; A61B 2090/395

(57) ABSTRACT

Methods and systems for generating 2D/3D images using dually synchronized pulsed lasers for a contactless ultrasound system. Directing two pulsed wave photoacoustic excitation sources working simultaneously and in synchronization into a desired area distributing acoustic energy into the tissue at the speed of sound. Contrary to a regular hand held ultrasound system, laser-generated ultrasonic waves have the dual advantage of non-contact and non-destructive application without requiring gel, water or electrodes application on the surface of the skin. Optical interferometric techniques are applied for detecting ultrasonic waves and the combination and synchronization of two photoacoustic excitation sources combined with external exteroceptive sensors allows for both post-processing and real-time view of images in a very efficient manner. The use of exteroceptive sensors may be used for image reconstruction and filtering techniques may be applied for 2D/3D image reconstruction and movement compensation.

### CROSS-REFERENCE TO RELATED APPLICATION

This application claims the benefit of the U.S. Provisional Application No. 63/303,410 entitled, “CONTACTLESS THREE DIMENSIONAL ELECTRO-MAGNETIC ULTRASOUND SYSTEM” filed on Jan. 26, 2022, which is incorporated herein by reference in its entirety.

### TECHNICAL FIELD

Embodiments relate generally to methods and systems to create 2D/3D ultrasound images completely contactless using two laser based ultrasonic sources working simultaneously and in synchronization in combination with external sensors.

### STATEMENT REGARDING FEDERALLY SPONSORED RESEARCH

N/A

### BACKGROUND OF THE INVENTION

The present invention relates to systems and methods for generating 2D and 3D ultrasound images without applying any contact to the surface, making the ultrasound system contact-less.

An ultrasound wave occurs at frequencies that are above the bandwidth of human audio capabilities. In terms of imaging, the frequencies cover a wide variety of applications, including, but not limited to, underwater sonar systems, industrial non-destructive testing, near-surface damage assessment, medical diagnostics, and evaluation of acoustic micro-structures.

Regular ultrasonic imaging techniques have long-known benefits. Technological advancements in hand-held probes have been proven to provide better images for soft tissue structures such as the eyes, abdomen, brain, neck, and feet. Conventional techniques for obtaining ultrasound images typically require applying a mechanical probe directly to the patient's body, focusing on the specific area of interest for a particular period of time. The amount of time spent probing is determined by the sonographer's level of experience and by the complexity of the anomaly being investigated.

During an ultrasound, the probe emits an acoustic wave, causing a thermal dilation of tissue that consequently deforms and elasticizes the tissue, generating a returning wave. These waves ricochet back to the probe, and the time difference between the original emission from the probe and the returning acoustic wave compose the sonogram, which is typically displayed on a monitoring system.

Unlike CT scans, MRIs, or x-rays, ultrasound imaging technologies are relatively low-cost and do not involve the injection of radiation or contrast. They are also portable. Ultrasound technicians and doctors must constantly balance the quality of scans with the hospitals' needs for efficient, high volume scanning to increase healthcare reimbursements.

The use of hand held probes, however, poses problems that current technology is not yet able to overcome. The evidence is clear that overuse injuries brought on by repetitive muscle stresses associated with the performance of ultrasound exams can lead to muscular damage and, in some cases, career-ending injuries. In addition, the hand-held probe may exacerbate existing pain for patients when technicians apply pressure on the point or area under investigation. This is especially prominent during a prolonged pressure application to a painful area.

Patients may also move during the procedure due to discomfort or pain, and this can interfere with the quality of the scan, corrupt the ultrasonic reading, or elongate the duration of the exam. If patients are in extreme pain or cannot be still during a scan, the hospital may have to reschedule a session, since efficiency and patient turnover must remain high in the current profit structure.

A patient's body type and size also impact the quality of an ultrasound. With overweight and obese patients, the ultrasound wave has to pass through more fat tissue before reaching the main site, sometimes causing an unclear reading. In addition, internal gas can also interfere with the accuracy of a reading, requiring a longer session. In these cases, the sonographer must apply a great deal of pressure to the site.

In order to produce an accurate reading using current ultrasound technology, the system must always have a clear understanding of where the probe is in space. Spatial resolution is often a major obstacle with handheld devices. Accurate readings require the system to have a continuous record of the angular velocity, linear velocity, roll, pitch, and yaw. While current technology offers spherical probe transducers, which concentrate the beam, there is still too much room for human error. For example, if the technician's hand trembles or if there is inconsistency in the amount of pressure they apply, this can compromise the integrity of the reading. In addition, if the sonographer did not apply enough gel to create the ideal electrodynamic balance, the returning wave could be flawed and lead to an inaccurate scan.

There is currently a significant amount of scientific research exploring image registration and feature matching when performing image reconstruction. Before an exam is even performed, all sensors must be calibrated so that images can be properly aligned during reconstruction. Researchers have also investigated the use of point cloud and motion compensation techniques to perform more accurate image reconstruction.

Based on the limitations of current technology, there is a need for more advanced point registration techniques and better 2D and 3D image reconstruction procedures. More advanced technology would lead to stronger decision making by physicians and higher quality diagnostic imagery. This is especially important because ultrasound technicians and doctors are constantly under the pressure of daily patient turnover and it is critical that the highest possible image quality is available when making high stakes and time-sensitive medical decisions.

Robotic-assisted surgical ablation currently utilizes ultrasounds to guide and support the procedures in real time. Because sterility is critical, there is an extensive and lengthy hospital procedure for ensuring that the probe is properly cleaned and prepared. This process must be repeated for each procedure in order to minimize the risk of infection, which is a drain on hospital resources.

Physician-led procedures, for example, liver biopsies or tumor ablation, that already utilize handheld ultrasound would also benefit from Artificial Intelligence-guided ultrasounds in order to reduce patient risk and improve outcomes.

Research and experimentation into pulse or continuous lasers to be used as contactless ultrasonic devices is in the initial stages. Some studies have proven that contactless ultrasound techniques can be a useful tool for both humans and animals. Eye-safe techniques are already being used to treat optical problems such as myopia and are considered safe at a particular exposure. Wireless portable ultrasound probes are already in use and present a great advantage in terms of portability and field operations. They do not, however, eliminate the need to physically touch the patient nor do they limit sonographers' stress injuries. This demonstrates a need for developing novel techniques in this field that allow for acoustic waves to enter soft tissue, skin, and organs without the need for physical contact with the patient.

### SUMMARY OF THE INVENTION

The following presents a simplified summary of the innovation in order to provide a basic understanding of some aspects of the invention. This summary is not an extensive overview of the invention. It is intended to neither identify key or critical elements of the invention nor delineate the scope of the invention. Its sole purpose is to present some concepts of the invention in a simplified form as a prelude to the more detailed description that is presented later. In order to overcome the aforementioned limitations in current ultrasound technology, the present invention utilizes simultaneous and synchronous laser-generated ultrasound waves combined with the use of exteroceptive sensors and filtering techniques.

The application of laser ultrasonics for clinical diagnostic assessment of the internal structure of tissues carries significant advantages over current models. A pulse laser, or Q-switched laser, may be used for internal localization and detection of features or soft tissues by emitting a photoacoustic ultrasonic wave toward the patient. The return wave is represented by a coherent summation of waves that present themselves on the patient's skin and are analyzed with an optical tool such as an interferometer. When the returning wave ricochets back to the surface it presents itself as a vibration at that specific point. That vibration corresponds to the maximum vibration detected at that point using the optical interferometer. The maximum vibrational point is then captured with the interferometer and given the proper xyz coordinate which will be properly assigned to a grid heatmap. The grid heatmap displays magnitude as color in a two-dimensional matrix. Each dimension represents a category of trait and the color represents the magnitude of specific measurement on the combined traits from each of the two categories. One dimension represents the length of the scanned area and the other dimension represents the width of the scanned area, and the value measured is the depth. This grid heat map would show how depth changes across different locations of the scanned area while the simultaneous and synchronous Q-switched laser device is moving according to a predefined area.

One embodiment of the present invention provides a method for generating 2D/3D ultrasound images of a patient using a single laser-based ultrasonic source. Unlike conventional ultrasonic efforts to establish the internal structure of soft tissues or organs, laser generation techniques, being contactless, do not require any specific preparation of the skin. The method includes generating ultrasonic acoustic waves using pulsed wave lasers. The beam of the excitation source is focused on a specific area of interest of the body and emits ultrasonic waves. The ultrasonic excitation source may include a lens apparatus able to direct and guide the beam.

Due to backscattering, returning acoustic waves are detected via interferometric systems. Detected waveforms on the skin of the patient are subsequently analyzed and processed to evaluate the internal structure. Exteroceptive sensors, such as a colored camera, a black and white camera, an Inertial Measurement Unit (IMU), a GPS, or an RTK GPS antenna, are used in combination with a robotic platform. Since a patient may move during the ultrasound session, the sonographer or the doctor can highlight specific points on the skin of the patient with a highlighter allowing the cameras to feature-detect those points and have a specific search area that keeps the reference frame stable.

Detected waveforms on the skin of the patient are subsequently analyzed by a processor which provides ultrasound images that may evaluate the internal structure. Ultimately, the processing step is directly connected to a data acquisition system that may be able to record the necessary data of all exteroceptive sensors.

Another embodiment of the present invention provides a method for generating 2D/3D ultrasound images of a patient using two laser-based ultrasonic sources simultaneously and in synchronization. The method includes generating ultrasonic acoustics from two different sources using pulsed lasers. The beams of the excitation sources are focused on a specific area of interest in the body and emit ultrasonic waves. The pulses of the lasers alternate each other. By way of example: Q-Switched laser A sends a pulse a time 0 at position 0 so the first wave can penetrate the surface of the skin and arrive at the preferred depth. Q-Switched laser B sends a pulse, for example, 10 nanoseconds later a time 1 at position 1 so the second wave can penetrate the surface of the skin and arrive at the preferred depth. The interval between the two pulses is when the maximum surface wave vibration is detected from the external interferometer. So in a continuous way, the system translates at extremely low speed while it performs the scan alternating the two pulses from the two different lasers. Additionally, in a continuous way, the system rotates at extremely low speed while it performs the scan alternating the two pulses from the two different lasers.

An additional embodiment of the present invention provides a method for generating 2D/3D ultrasound images of a patient. The method stores data points from the lasers and all exteroceptive sensor positions and orientations in an information log upon indication of the sonographer or the doctor. The stored data points are used to build a list of vector positions and orientations which can then be used to play-back the recorded session, train sonographers or doctors or further studies.

Still another embodiment of the present invention provides a method for generating 2D/3D ultrasound images of a patient by displaying an ultrasound view of an anatomic structure derived from two laser-based ultrasonic sources working simultaneously and in synchronization. The system works by applying the concept of superposition to overlap layers of images taken from the various cameras and lasers, aligning with the position and orientation of the exteroceptive sensors in order to accurately reconstruct the images. This embodiment can be divided into the following steps: a) since a patient may move during the ultrasound session, the sonographer or the doctor can highlight specific points on the skin of the patient with a highlighter allowing the cameras to feature-detect those points and have a specific search area that keeps the reference frame stable; b) the two laser-based ultrasonic sources working simultaneously and in synchronization start emitting pulses alternating laser A and laser B on the specific area mentioned at point a) allowing for surface wave maximum detection; c) the interferometer detects those maximum vibrational waves within the area and assign xyz location to every point; d) all those points are combined together to create a heat grid map at that specific depth; e) laser A and laser B are regulated to have a higher or lower penetration rate so it is possible to create another layer (another grid map) at a different depth but within the same area mentioned at point a); f) repeat process e) for several depth so that the sonographer can form different layers at different depths of the seame search area; g) interpolate the different layers in order to form a 3D image completely contactless.

Knowledge of the acoustic wavelengths between different structures of the body allows for internal structures to be reconstructed, therefore between 1400 nanometers (nm) and 1600 nanometers (nm) are the preferred emitting ranges of the pulsed lasers.

Additionally, compensation techniques used for image reconstruction may be derived from non-parametric filtering techniques, included but not limited to, histogram filters, particle filters and gaussian probability hypothesis density filters.

Additional features and advantages of the various aspects of the present invention become apparent from the following description of its preferred embodiments. The description provided should be taken in consideration with the accompanying drawings. The aforementioned embodiments do not represent the full scope of the invention. The references following the description of the preferred embodiments are made thereof to the claims and herein for interpreting the scope of the present invention.

Other objects, features and advantages of the invention shall become apparent as the description thereof proceeds when considered in connection with the accompanying illustrative drawings.

### BRIEF DESCRIPTION OF THE DRAWINGS

The novel features that are characteristic of the present invention are set forth in the appended claims. However, the invention's preferred embodiments, together with further objects and attendant advantages, will be best understood by reference to the following detailed description taken in connection with the accompanying Figures:

FIG. 1 is an image showing a system configured to implement the present invention for 3D reconstruction of images.

FIG. 2 is an image showing a portable system using a remote excitation source configured to implement the present invention for 3D reconstruction of images.

FIG. 3 is an image showing a system configured to implement the present invention for 3D reconstruction of images.

FIG. 4 is an image showing a portable system configured to implement the present invention for 3D reconstruction of images.

FIG. 5 is an image showing a system configured to implement the present invention for 3D reconstruction of images. The system translates in xyz direction by way of stepping motors.

FIG. 6 is an image showing a portable system configured to implement the present invention for 3D reconstruction of images.

FIG. 7 is an image showing a system configured to implement the present invention for 3D reconstruction of images. The system translates in xyz direction by way of stepping motors and may rotate around the patient by way of stepping motors.

FIG. 8 is an image showing a portable system configured to implement the present invention for 3D reconstruction of images.

FIG. 9 is an image showing a system configured to implement the present invention for 3D reconstruction of images.

FIG. 10 is an image showing a portable system configured to implement the present invention for 3D reconstruction of images.

FIG. 11 is an image showing a system configured to implement the present invention for 3D reconstruction of images. The system translates in xyz direction by way of stepping motors and may rotate around the patient by way of stepping motors.

FIG. 12 is an image showing a portable system configured to implement the present invention for 3D reconstruction of images.

FIG. 13 is a flow chart explaining the post-processing procedure and image reconstruction for a single laser case.

FIG. 14 is a flow chart explaining the post-processing procedure and image reconstruction for a single laser case with an additional step of matching stereo pair images and laser images.

FIG. 15 is a flow chart explaining the post-processing procedure and image reconstruction for two photoacoustic excitation sources working simultaneously and in synchronization.

FIG. 16 is a flow chart explaining the post-processing procedure and image reconstruction for two photoacoustic excitation sources working simultaneously and in synchronization with an additional step of matching stereo pair images and laser images.

FIG. 17 is a flow chart explaining the real-time processing procedure and image reconstruction for a single laser case.

FIG. 18 is a flow chart explaining the real-time processing procedure and image reconstruction for a single laser with an additional step of matching stereo pair images and laser images.

FIG. 19 is a flow chart explaining the real-time processing procedure and image reconstruction for two photoacoustic excitation sources working simultaneously and in synchronization.

FIG. 20 is a flow chart explaining the real-time processing procedure and image reconstruction for two photoacoustic excitation sources working simultaneously and in synchronization with an additional step of matching stereo pair images and laser images.

FIG. 21 is a flow chart explaining the real-time processing procedure and image reconstruction for two photoacoustic excitation sources working simultaneously and in synchronization and interferometric system inputting vibrometry data into the external visualizer.

FIG. 22 is a flow chart explaining the real-time processing procedure and image reconstruction for two photoacoustic excitation sources working simultaneously and in synchronization and interferometric system inputting vibrometry data into the filtering system after being subjected to transformation and compensation of reference frames.

FIG. 23 is a flow chart explaining the post-processing procedure and image reconstruction for two photoacoustic excitation sources working simultaneously and in synchronization subjected to further compensation of reference frames.

FIG. 24 is a flow chart explaining the post-processing procedure and image reconstruction for a single laser and a single interferometer subjected to further compensation of reference frames.

FIG. 25 is a flow chart explaining the post-processing procedure and image reconstruction for two photoacoustic excitation sources working simultaneously and in synchronization and an interferometric system subjected to further compensation of reference frames.

FIG. 26 is a flow chart explaining the post-processing procedure and image reconstruction for a single laser subjected to further compensation of reference frames.

### DETAILED DESCRIPTION OF THE INVENTION

The subject innovation is now described with reference to the drawings, wherein like reference numerals are used to refer to like elements throughout. In the following description, for purposes of explanation, numerous specific details are set forth in order to provide a thorough understanding of the present invention. It may be evident, however, that the present invention may be practiced without these specific details. In other instances, well-known structures and devices are shown in block diagram form in order to facilitate describing the present invention.

For convenience, the meaning of some terms and phrases used in the specification, examples, and appended claims, are listed below. Unless stated otherwise or implicit from context, these terms and phrases have the meanings below. These definitions are to aid in describing particular embodiments and are not intended to limit the claimed invention. Unless otherwise defined, all technical and scientific terms have the same meaning as commonly understood by one of ordinary skills in the art to which this invention belongs. For any apparent discrepancy between the meaning of a term in the art and a definition provided in this specification, the meaning provided in this specification shall prevail.

“Data Acquisition System (DAS)” has the electronic hardware art-defined meaning. A DAS for the context of this patent is intended to be an oscilloscope which is able to process data originating from the backscattering of ultrasonic waveforms on the surface of the tissue.

“Data Log (DL)” has the database art-defined meaning. A DL for the context of this patent is intended to be a storage system such as a database, where all information from external sensors are continuously stored in real-time. Such information is, for example, displacement, position, velocity and acceleration.

“External Sensors (ES)” has the hardware art-defined meaning. The external sensors for the context of this patent are defined as the two lasers, the interferometric receiving system (receiver device), the GPS, the color camera, the black and white camera and the stepping motors. There is a 3-axis IMU mounted on each of these sensors.

“External Visualizer (EV)” has the hardware art-defined meaning. The EV is intended as a regular computer monitor. A 3D visualizer is also a computer monitor where to show the 3D image. For the context of this patent external visualizer and 3D visualizer are intended as regular computer monitor and these terms will be used interchangeably.

“Filtering Techniques (FT)” has the mathematical robotic art-defined meaning. FT processes are algorithms aimed at finding patterns in historical and current data to extend them into future predictions, providing insights into what might happen next. Filtering techniques can be divided into parametric and non-parametric as explained below.

“Global Positioning System (GPS)” has the mathematical robotic art-defined meaning. GPS is a radio navigation system that allows land, sea, and airborne users to determine their exact location, velocity, and time 24 hours a day, in all conditions, in every instant of time. A GPS system can be considered as the absolute reference frame system from which local relative reference frames refer to while in movement.

“Grid Heatmap (GH)” has the mathematical art-defined meaning. The GH displays magnitude as color in a two-dimensional matrix. Each dimension represents a category of trait and the color represents the magnitude of specific measurement on the combined traits from each of the two categories. One dimension represents the length of the scanned area and the other dimension represents the width of the scanned area, and the value measured is the depth. This grid heat map would show how depth changes across different locations of the scanned area under investigation. The variation in color may be by hue or intensity, giving obvious visual cues to the user about how the phenomenon varies over space.

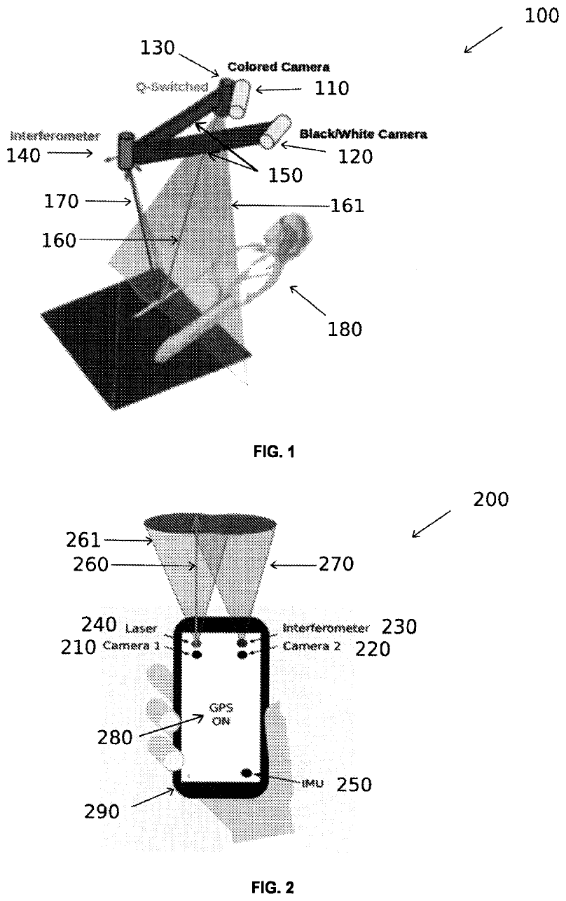

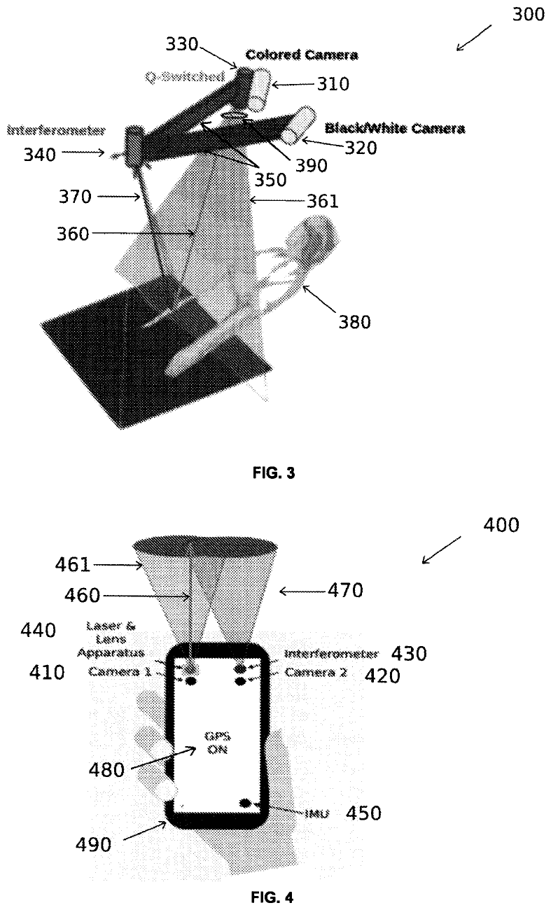

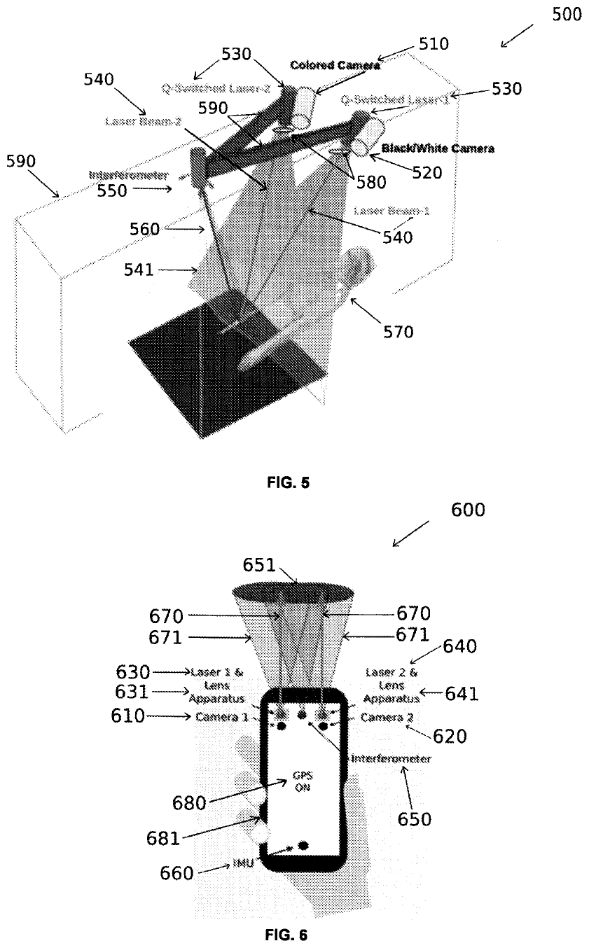

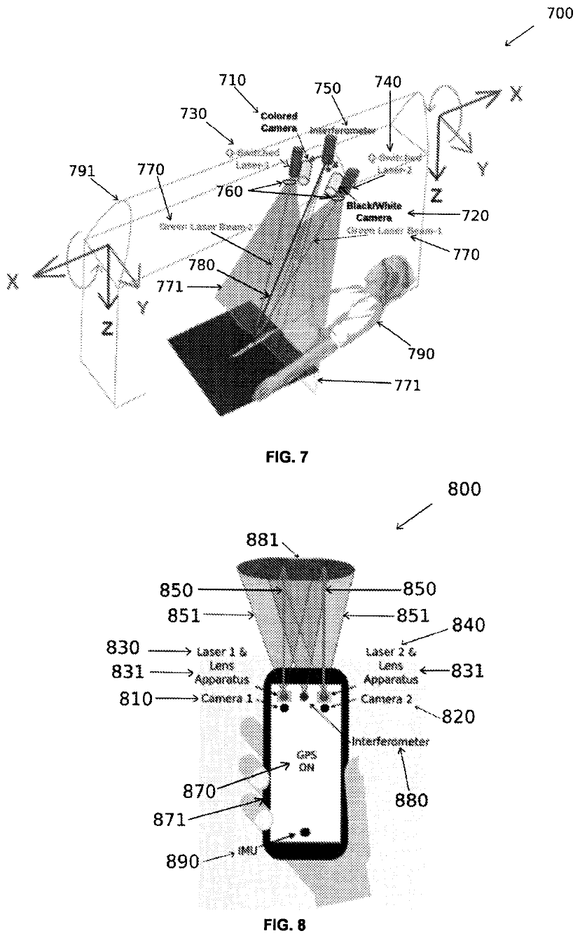

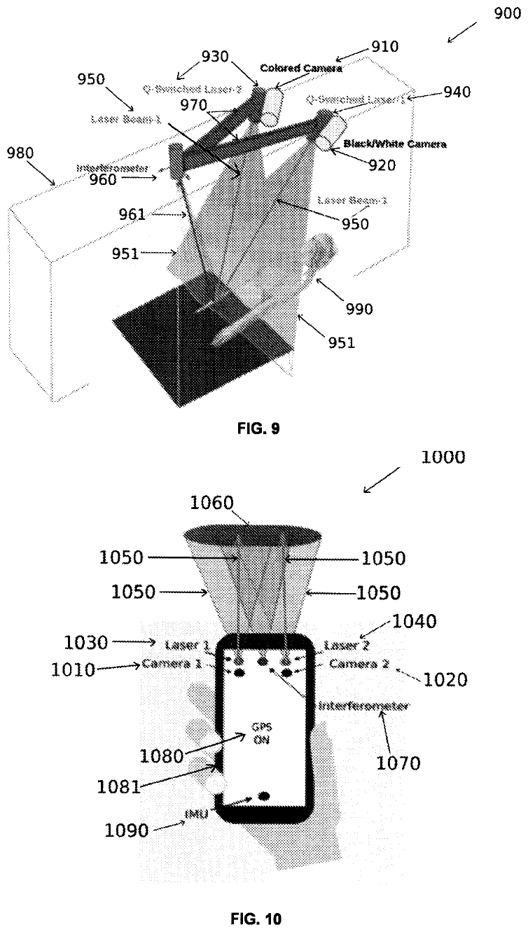

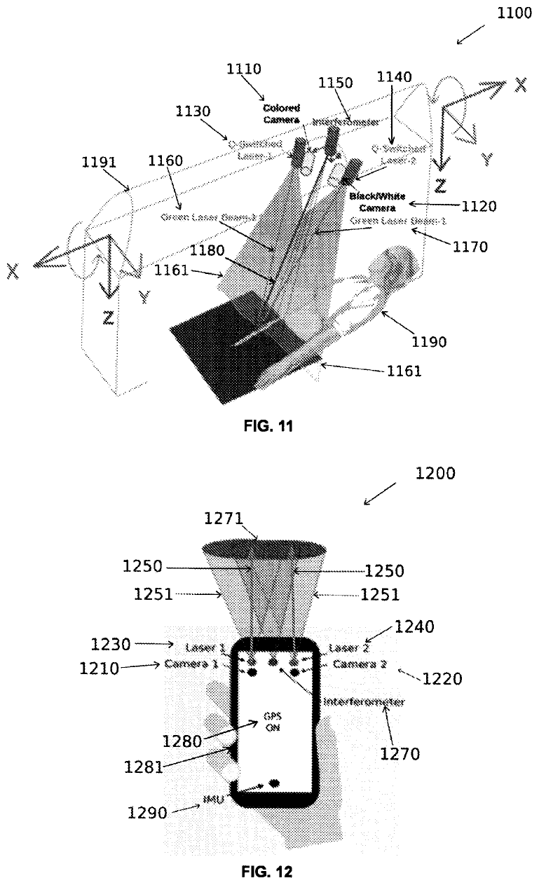

“Inertial Measurement Unit (IMU)” has the mathematical robotic art-defined meaning. IMUs are inertial sensors commonly used in robotics motion, terrain compensation, platform stabilization, antenna and camera pointing applications. An individual IMU can sense a measurement along or about a single axis. To provide a three-dimensional solution, three individual IMUs must be mounted together into an orthogonal cluster. This set of IMUs mounted is commonly referred to as a 3-axis inertial sensor, as the sensor can provide one measurement along each of the three axis. Therefore, in this patent every sensor listed (colored camera, black and white camera, GPS, or RTK GPS antenna, Q-switched laser(s) and interferometer) will be equipped with a 3-axis IMU so that measurement on each direction (roll-pitch-yaw) is available at any time and in every instant; specifically: FIG. 1, FIG. 3, FIG. 5, FIG. 7, FIG. 9 and FIG. 11. Additionally when referring to the 3-axis IMU mounted on a portable device such as FIG. 2, FIG. 4, FIG. 6, FIG. 8, FIG. 10 and FIG. 12, one single 3-axis IMU sensor will be mounted on the portable device.

“Information Logger (IL)” has the database art-defined meaning. An IL for the context of this patent is intended to be a storage system such as a database, where all information from external sensors are continuously stored. Such information are, for example, displacement, position, velocity and acceleration.

“Kalman Filter” has the mathematical art-defined meaning. The Kalman Filter is an optimal estimator defined as a set of mathematical equations that provides a recursive computational methodology for estimating the state of a discrete process from measurements that are typically noisy, while providing an estimate of the uncertainty of the estimates. Kalman Filter uses linear approximations of the state and measurement models to generate a state estimate given a prior state and associated uncertainties.

“Laser Stereometry (LS)” has the mathematical robotic art-defined meaning. A laser stereo image contains two views of a scene side by side. One laser view is intended for the left eye and the other laser view for the right eye.

“Non-Parametric Filter” has the mathematical robotic art-defined meaning. Non-parametric filters do not rely on any specific parameter settings and therefore tend to produce more accurate results. Non-parametric filters approximate posteriors by a finite number of values, each roughly corresponding to a region in state space. Examples of non-parametric filters are: histogram filter, particle filter, PHD filter, gaussian mixture PHD filter.

“Odometry” has the mathematical robotic art-defined meaning. Odometry is a common method of determining an object's motion from the way in which subsequent images overlap.

“Particle Filter (PF)” has the mathematical robotic art-defined meaning. The PF is a non-parametric solution to the Bayes Filter which uses a set of samples or “particles”. Each one of these particles can be seen as the possibility of an object being at that position at that time. PF is divided into three main steps. Prediction or state transition model, where the particle's position is predicted according to external sensors. Weighting or measurement model, where particles are weighted according to their likelihood with data provided by external sensors, and finally, resample, where particles with small weights are discarded.

“Processing System (PS)” has the hardware art-defined meaning. PS or Processor System is a computer such as a laptop or tower desktop able to process data in real-time or post-processing or pre-processing originated from a scanning session.

“Robotic Platform (RP)” has the robotic art-defined meaning. A RP is a set of open-source software frameworks, libraries, and tools to create applications for robots such as the Robotic Operating System (ROS). A RP provides a range of features that come with a standard operating system such as hardware abstraction and sensor integration package management. Additionally, a RP allows the user to develop customized software and processes to enhance existing features of sensors. All sensors listed in this patent can be integrated into a RP such as IMUs, Interferometers, Q-Switched Lasers, Cameras (colored and black and white) and stepping motors.

“Posterior” has the statistics art-defined meaning. A posterior, also known as posterior probability, in Bayesian statistics, is the revised or updated probability of an event occurring after taking into consideration new historical information in order to predict what might happen next. The posterior probability is calculated by updating the prior probability using Bayes' theorem. The posterior probability is the probability of event A occurring given that event B has occurred.

“Prior” has the statistics art-defined meaning. A Prior also known as prior probability, in Bayesian statistics, is the probability of an event occurring before new (posterior) data is collected.

“Real-Time Mapper (RTM)” has the mathematical robotic art-defined meaning. In order to properly position images in real-time on the correct reference frame a rotation matrix transformation from Real-Time Mapper to Ultrasound Reference frame is needed. The RTM is in charge of correctly showing the projection of the heat grid map in the real-time reference frames while the system is scanning the patient.

“Stereo Images (SI)” has the mathematical robotic art-defined meaning. A stereo image contains two views of a scene side by side. One of the views is intended for the left eye and the other for the right eye. A stereo pair of images taken at each view's location is used to find distance. Then, the image from one camera is registered to the same camera's previous image, correcting rotation and scale differences. This registration is used to find the area's motion and speed between imaging positions.

“Ultrasound Laser System Scanning Session Data (ULSSSD)” has the robotic art-defined meaning. The ULSSSD is the main container of the recorded session. All necessary data such as laser sources, external sensors such as cameras, 3-axis IMU, vibrometric sensor and a GPS, or an RTK GPS antenna system are recorded in the ULSSSD. ULSSSD works both real-time and post-processing.

“Vibrometric Sensor (VS)” has the robotic art-defined meaning. For the context of this patent a VS or interferometer have the same meaning. These instruments are able to optically detect surface vibrations. Therefore, these terms will be used interchangeably in this patent.

“Visual Inertial Odometry (VIO)” has the mathematical robotic art-defined meaning. VIO is a common method of determining an object's motion from the way in which subsequent images overlap.

EXAMPLE 1: System with single source Q-Switched pulsed laser, colored camera, black and white camera, interferometer, GPS and 3-axis IMU.

FIG. 1 illustrates an exemplary system 100 configured to implement the present invention for 3D reconstruction of images.

Images taken from a colored camera 110 and a black and white camera 120 are used to form stereo images and provide a 3D external shape of the patient 180. Patient's movement is compensated via real-time visual inertial odometry. Stereo images are used during the external reconstruction of the human shape providing a state of the art measured image. Knowing the position of the two cameras allows precise measurement of the patient 180. The 3-axis IMU used for real-time position and orientation integrated in the structure 150 is not visible but is present. Additionally a 3-axis IMU is integrated in the GPS (not visible but is present in the structure 150), or an RTK GPS antenna, in every Q-Switched laser, in the colored camera, in the black and white camera and in the interferometer. Since a patient may move during the ultrasound session, the sonographer or the doctor can highlight specific points on the skin of the patient with a highlighter allowing the cameras to feature-detect those points and have a specific search area box.

A Q-switched pulsed laser 130 emits an ultrasonic wave on top of the human body, or on a preferred area, that will propagate in the internal part of the body. Through a backscatter coherent summation of the return waves up to the skin of the patient, it is possible to detect such displacement via an interferometric system 140. Laser 130 operates simultaneously and in synchronization with the other external sensors.

In a hospital setting system 100 fits all around the patient and all the external sensors such as Q-Switched pulsed laser, colored camera, black and white camera, interferometer, GPS are present. All the sensors can be fixed while the platform 150 moves (translates) accordingly after the input of the doctor's or the sonographer's about the part to scan. Alternatively, the sensors remain fixed and the system 100 translates and rotates accordingly on top of the search area decided by the doctor by way of stepping motors.

The ultrasonic wave is emitted in the wavelength where the absorption is preferred by the human body and its internal tissues or organs in order to have an optimal response.

The interferometer 140 detects those maximum vibrational waves within the area the doctor's or the sonographer's decided to scan and assign xyz location to every point. All those points are combined together to create a heat grid map at that specific depth.

170 simply represents an interferometer observing the maximum vibration detected on the skin at a specific point. Returning waves on the skin outside of the field of view of the interferometer will only give a partial or incomplete reading. Therefore, it is important that the field of view of the laser 161 and the field of view of the interferometer overlap for optimal detection.

The laser is regulated to have a higher or lower penetration rate so it is possible to create another layer (another grid map) at a different depth but within the same investigational area mentioned above. Repeating the scanning process for several depths will provide the sonographer with different layers of the preferred area of investigation. At this point it is possible to simply interpolate the different layers in order to form a 3D image completely contactless.

Additional real-time grid map construction can be built using filtering techniques, specifically using non-parametric filters such as particle filter techniques.

EXAMPLE 2: Portable system.

In case of an exemplary handheld device as shown in FIG. 2, the system 200 and the same sensors are used such as a colored camera 210 and a black and white camera 220, the 3-axis IMU 250, the GPS 280, or RTK GPS antenna, for real-time positioning, the Q-switched laser 240 and the interferometer 230. 261 simply represents the field of view of the laser.

The 3-axis IMU 250 is able to provide a precise orientation together with linear and angular velocities being able to detect the smallest movement in roll, pitch and yaw.

The 3-axis IMU 250 integrates multi-axis, accelerometers and gyroscopes altogether to provide an object's orientation in space. Together the emitting laser beam 260 and the external interferometer 230 are able to detect the receiving ultrasonic waves as a result of coherent summation on the skin. 270 simply represents the field of view of the interferometer. Returning waves on the skin outside of the field of view of the interferometer will only give a partial or incomplete reading. Therefore, it is important that the field of view of the laser 261 and the field of view of the interferometer 270 overlap for optimal detection. The 3D images are visualized on a portable device 290 remote from the patient that enables real-time functioning. The interferometer 230 detects those maximum vibrational waves within the search area established by the sonographer or the doctor and assign xyz location to every vibrational point detected. All those points are combined together to create a heat grid map at that specific depth. The laser is regulated to have a higher or lower penetration rate so it is possible to create another layer (another grid map) at a different depth but within the same investigational area mentioned above. Repeating the scanning process for several depths will provide the sonographer with different layers of the preferred area of investigation. At this point it is possible to simply interpolate the different layers in order to form a 3D image completely contactless.

Additional real-time grid map construction can be built using filtering techniques, specifically using non-parametric filters such as particle filter techniques.

EXAMPLE 3: System with single source Q-Switched pulsed laser, colored camera, black and white camera, interferometer, GPS, 3-axis IMU and lens apparatus.

FIG. 3 illustrates an exemplary system 300 configured to implement the present invention for 3D reconstruction of images using lens apparatus able to guide the ultrasonic emitting beam 360. 361 simply represents the field of view of the laser.

Images taken from a colored camera 310 and a black and white camera 320 are used to form stereo images and provide a 3D external shape of the patient 380. Patient's movement is compensated via real-time visual inertial odometry. Stereo images are used during the external reconstruction of the human shape providing a state of the art measured image. Knowing the position of the two cameras allows precise measurement of the patient 380. The 3-axis IMU used for real-time position and orientation integrated in the structure 350 is not visible but is present. Additionally a 3-axis IMU is integrated in the GPS (not visible but is present in the structure 350), or an RTK GPS antenna, in every Q-Switched laser, in the colored camera, in the black and white camera and in the interferometer. Since a patient may move during the ultrasound session, the sonographer or the doctor can highlight specific points on the skin of the patient with a highlighter allowing the cameras to feature-detect those points and have a specific search area box.

A Q-switched pulsed laser 330 emits an ultrasonic wave on top of the human body, or on a preferred area, that will propagate in the internal part of the body. Through a backscatter coherent summation of the return waves up to the skin of the patient, it is possible to detect such displacement via an interferometric system 340. Laser 330 operates simultaneously and in synchronization with the other external sensors.

In a hospital setting system 300 fits all around the patient and all the external sensors such as Q-Switched pulsed laser, colored camera, black and white camera, interferometer, GPS are present. All the sensors can be fixed while the platform 150 moves (translates) accordingly after the input of the doctor's or the sonographer's about the part to scan. Alternatively, the sensors remain fixed and the system translates and rotates accordingly on top of the search area decided by the doctor and the lens apparatus will guide the beam by way of stepping motors.

The ultrasonic wave is emitted in the wavelength where the absorption is preferred by the human body and its internal tissues or organs in order to have an optimal response.

The interferometer 340 detects those maximum vibrational waves within the area the doctor's or the sonographer's decided to scan and assign xyz location to every point. All those points are combined together to create a heat grid map at that specific depth.

370 simply represents an interferometer observing the maximum vibration detected on the skin at a specific point. Returning waves on the skin outside of the field of view of the interferometer will only give a partial or incomplete reading. Therefore, it is important that the field of view of the laser 361 and the field of view of the interferometer overlap for optimal detection.

The laser is regulated to have a higher or lower penetration rate so it is possible to create another layer (another grid map) at a different depth but within the same investigational area mentioned above. Repeating the scanning process for several depths will provide the sonographer with different layers of the preferred area of investigation. At this point it is possible to simply interpolate the different layers in order to form a 3D image completely contactless.

The lens apparatus 390 is able to guide the ultrasonic emitting beam. The lens apparatus 390 can also be used to guide the laser beams in a specific area of the body. Between two different laser pulses is the moment where the maximum vibrational point is optically observed by the interferometer on the skin. That point is assigned specific xyz coordinates that will constitute a heat grid map. The laser beam may move, or be guided through the lens apparatus, on top of the search area established by the doctor or the sonographer and move according to specific patterns such as lawn-mower.

Additional real-time grid map construction can be built using filtering techniques, specifically using non-parametric filters such as particle filter techniques.

EXAMPLE 4: Portable system.

In case of an exemplary handheld device as shown in FIG. 4, the system 400 and the same sensors are used such as a colored camera 410 and a black and white camera 420, the 3-axis IMU 450, the GPS 480, or RTK GPS antenna, for real-time positioning, the Q-switched laser 460 and the interferometer 430. 461 simply represents the field of view of the laser.

The 3-axis IMU 450 is able to provide a precise orientation together with linear and angular velocities being able to detect the smallest movement in roll, pitch and yaw.

The 3-axis IMU 450 integrates multi-axis, accelerometers and gyroscopes altogether to provide an object's orientation in space. Together the emitting laser beam 460 and the external interferometer 430 are able to detect the receiving ultrasonic waves as a result of coherent summation on the skin. Returning waves on the skin outside of the field of view of the interferometer will only give a partial or incomplete reading. Therefore, it is important that the field of view of the laser 461 and the field of view of the interferometer 470 overlap for optimal detection. The 3D images are visualized on a portable device 490 remote from the patient that enables real-time functioning. The interferometer 430 detects those maximum vibrational waves within the search area established by the sonographer or the doctor and assign xyz location to every vibrational point detected. All those points are combined together to create a heat grid map at that specific depth. The laser is regulated to have a higher or lower penetration rate so it is possible to create another layer (another grid map) at a different depth but within the same investigational area mentioned above. Repeating the scanning process for several depths will provide the sonographer with different layers of the preferred area of investigation. At this point it is possible to simply interpolate the different layers in order to form a 3D image completely contactless.

The lens apparatus 440 is able to guide the ultrasonic emitting beam. The lens apparatus 440 can also be used to guide the laser beams in a specific area of the body.

Between two different laser pulses is the moment where the maximum vibrational point is optically observed by the interferometer on the skin. That point is assigned specific xyz coordinates that will constitute a heat grid map. The laser beam may move, or be guided through the lens apparatus, on top of the search area established by the doctor or the sonographer and move according to specific patterns such as lawn-mower.

Additional real-time grid map construction can be built using filtering techniques, specifically using non-parametric filters such as particle filter techniques.

EXAMPLE 5: Simultaneous and synchronized Q-Switched pulsed lasers, colored camera, black and white camera, interferometer, GPS, 3-axis IMU and lens apparatus.

FIG. 5 illustrates an exemplary system 500 configured to implement the present invention for 3D reconstruction of images with two excitation sources 530 configured to be implemented into the system working simultaneously and in synchronization. Additionally there is with a lens apparatus 580 able to guide the ultrasonic emitting beams 540 together with the external sensors such as a colored camera 510 and a black and white camera 520, the 3-axis IMU (integrated in the structure 590), the GPS (integrated in the structure 590), or RTK GPS antenna, for real-time positioning and the interferometer 550. 541 simply represents the field of view of the laser.

Images taken from a colored camera 510 and a black and white camera 520 are used to form stereo images and provide a 3D external shape of the patient 570. Patient's movement is compensated via real-time visual inertial odometry. Stereo images are used during the external reconstruction of the human shape providing a state of the art measured image. Knowing the position of the two cameras allows precise measurement of the patient 570. The 3-axis IMU used for real-time position and orientation integrated in the structure 590 is not visible but is present. Additionally a 3-axis IMU is integrated in the GPS (not visible but is present in the structure 590), or an RTK GPS antenna, in every Q-Switched laser 530, in the colored camera 510, in the black and white camera 520 and in the interferometer 550. Since a patient may move during the ultrasound session, the sonographer or the doctor can highlight specific points on the skin of the patient with a highlighter allowing the cameras to feature-detect those points and have a specific search area box.

Two Q-switched pulsed lasers 530 emit an ultrasonic wave on top of the human body, or on the preferred part, that will propagate in the internal part of the body. Through a backscatter coherent summation of the return waves up to the skin of the patient it is possible to detect such displacement via an interferometric system 550. Lasers 530 operate simultaneously and in synchronization with the other external sensors.

In a hospital setting system 500 fits all around the patient and all the external sensors such as Q-Switched pulsed laser, colored camera, black and white camera, interferometer, GPS are present. All the sensors can be fixed while the platform 590 moves (translates) accordingly after the input of the doctor's or the sonographer's about the part to scan. Alternatively, the sensors remain fixed and the system translates and rotates accordingly on top of the search area decided by the doctor and the lens apparatus will guide the beam by way of stepping motors.

The ultrasonic wave is emitted in the wavelength where the absorption is preferred by the human body and its internal tissues or organs in order to have an optimal response.

The interferometer 550 detects those maximum vibrational waves within the area the doctor's or the sonographer's decided to scan and assign xyz location to every point. All those points are combined together to create a heat grid map at that specific depth.

560 simply represents an interferometer observing the maximum vibration detected on the skin at a specific point. Returning waves on the skin outside of the field of view of the interferometer will only give a partial or incomplete reading. Therefore, it is important that the field of view of the two lasers 541 and the field of view of the interferometer overlap for optimal detection.

The lasers are regulated to have a higher or lower penetration rate so it is possible to create another layer (another grid map) at a different depth but within the same investigational area mentioned above. Repeating the scanning process for several depths will provide the sonographer with different layers of the preferred area of investigation. At this point it is possible to simply interpolate the different layers in order to form a 3D image completely contactless.

The lens apparatus 580 is able to guide the ultrasonic emitting beam. The lens apparatus 580 can also be used to guide the laser beams in a specific area of the body.

Between two different laser pulses from the two laser sources is the moment where the maximum vibrational point is optically observed by the interferometer on the skin. That point is assigned specific xyz coordinates that will constitute a heat grid map. The laser beams may move, or be guided through the lens apparatus, on top of the search area established by the doctor or the sonographer and move according to specific patterns such as lawn-mower.

Additional real-time grid map construction can be built using filtering techniques, specifically using non-parametric filters such as particle filter techniques.

EXAMPLE 6: Portable system.

In case of an exemplary handheld device as shown in FIG. 6, the system 600 and the same sensors are used such as a colored camera 610 and a black and white camera 620, the 3-axis IMU 660, GPS 680, or RTK GPS antenna, for real-time positioning, the Q-switched laser 630 and 640 with their lens apparatuses 631 and 641 and the interferometer 650. 670 simply represents the field of view of the lasers.

The 3-axis IMU 660 is able to provide a precise orientation together with linear and angular velocities being able to detect the smallest movement in roll, pitch and yaw.

The 3-axis IMU 660 integrates multi-axis, accelerometers and gyroscopes altogether to provide an object's orientation in space. Together the emitting laser beam 670 and the external interferometer 650 are able to detect the receiving ultrasonic waves as a result of coherent summation on the skin. The 3D images are visualized on a portable device 681 remote from the patient that enables real-time functioning. The interferometer 650 detects those maximum vibrational waves within the search area established by the sonographer or the doctor and assign xyz location to every vibrational point detected. All those points are combined together to create a heat grid map at that specific depth. The lasers are regulated to have a higher or lower penetration rate so it is possible to create another layer (another grid map) at a different depth but within the same investigational area mentioned above. Repeating the scanning process for several depths will provide the sonographer with different layers of the preferred area of investigation. At this point it is possible to simply interpolate the different layers in order to form a 3D image completely contactless.

The lens apparatus 631 and 641 is able to guide the two ultrasonic emitting beams 670. The lens apparatus 631 and 641 can also be used to guide the laser beams 670 in a specific area of the body.

Between two different laser pulses from the two laser sources is the moment where the maximum vibrational point is optically observed by the interferometer on the skin. That point is assigned specific xyz coordinates that will constitute a heat grid map. The laser beams may move, or be guided through the lens apparatus, on top of the search area established by the doctor or the sonographer and move according to specific patterns such as lawn-mower.

Additional real-time grid map construction can be built using filtering techniques, specifically using non-parametric filters such as particle filter techniques.

EXAMPLE 7: Simultaneous and synchronized Q-Switched pulsed laser, colored camera, black and white camera, interferometer, GPS, 3-axis IMU and lens apparatus. The system is able to rotate and translate around the patient.

FIG. 7 illustrates an exemplary system 700 configured to implement the present invention for 3D reconstruction of images with two excitation sources 730 and 740 configured to be implemented into the system working simultaneously and in synchronization using a lens apparatus 760 able to guide the ultrasonic emitting beams 770 together with the external sensors such as a colored camera 710, a black and white camera 720, the 3-axis IMU (integrated in the structure 791), the GPS (integrated in the structure 791), or RTK GPS antenna, for real-time positioning and the interferometer 750. 771 simply represents the field of view of the lasers.

Images taken from a colored camera 710 and a black and white camera 720 are used to form stereo images and provide a 3D external shape of the patient 790. Patient's movement is compensated via real-time visual inertial odometry. Stereo images are used during the external reconstruction of the human shape providing a state of the art measured image. Knowing the position of the two cameras allows precise measurement of the patient 790. The 3-axis IMU used for real-time position and orientation integrated in the structure 791 is not visible but is present. Additionally a 3-axis IMU is integrated in the GPS (not visible but is present in the structure 791), or an RTK GPS antenna, in every Q-Switched laser 730 and 740, in the colored camera 710, in the black and white camera 720 and in the interferometer 750. Since a patient may move during the ultrasound session, the sonographer or the doctor can highlight specific points on the skin of the patient with a highlighter allowing the cameras to feature-detect those points and have a specific search area.

Two Q-switched pulsed lasers 730 and 740 emit an ultrasonic wave on top of the human body, or on the preferred part, that will propagate in the internal part of the body. Through a backscatter coherent summation of the return waves up to the skin of the patient it is possible to detect such displacement via an interferometric system 750. Lasers 730 and 740 operate simultaneously and in synchronization with the other external sensors.

In a hospital setting system 700 fits all around the patient and all the external sensors such as Q-Switched pulsed laser, colored camera, black and white camera, interferometer, GPS are present. All the sensors can be fixed while the platform 791 moves (translates) accordingly after the input of the doctor's or the sonographer's about the part to scan. Alternatively, the sensors remain fixed and the system translates and rotates accordingly on top of the search area decided by the doctor and the lens apparatus will guide the beam by way of stepping motors.

The ultrasonic wave is emitted in the wavelength where the absorption is preferred by the human body and its internal tissues or organs in order to have an optimal response.

The interferometer 750 detects those maximum vibrational waves within the area the doctor's or the sonographer's decided to scan and assign xyz location to every point. All those points are combined together to create a heat grid map at that specific depth.

780 simply represents an interferometer observing the maximum vibration detected on the skin at a specific point. Returning waves on the skin outside of the field of view of the interferometer will only give a partial or incomplete reading. Therefore, it is important that the field of view of the two lasers 771 and the field of view of the interferometer overlap for optimal detection.

The lasers are regulated to have a higher or lower penetration rate so it is possible to create another layer (another grid map) at a different depth but within the same investigational area mentioned above. Repeating the scanning process for several depths will provide the sonographer with different layers of the preferred area of investigation. At this point it is possible to simply interpolate the different layers in order to form a 3D image completely contactless.

The lens apparatus 760 is able to guide the ultrasonic emitting beam. The lens apparatus 760 can also be used to guide the laser beams in a specific area of the body.

Between two different laser pulses from the two laser sources is the moment where the maximum vibrational point is optically observed by the interferometer on the skin. That point is assigned specific xyz coordinates that will constitute a heat grid map. The laser beams may move, or be guided through the lens apparatus, on top of the search area established by the doctor or the sonographer and move according to specific patterns such as lawn-mower.

Additional real-time grid map construction can be built using filtering techniques, specifically using non-parametric filters such as particle filter techniques.

EXAMPLE 8: Portable system.

In case of an exemplary handheld device as shown in FIG. 8, the system 800 and the same sensors are used such as a colored camera 810 and a black and white camera 820, the 3-axis IMU 890, GPS 870, or RTK GPS antenna, for real-time positioning, the Q-switched laser 830 and 840 with their lens apparatuses 831 and the interferometer 880. 851 simply represents the field of view of the lasers.

The 3-axis IMU 890 is able to provide a precise orientation together with linear and angular velocities being able to detect the smallest movement in roll, pitch and yaw.

The 3-axis IMU 890 integrates multi-axis, accelerometers and gyroscopes altogether to provide an object's orientation in space. Together the emitting laser beams 850 and the external interferometer 880 are able to detect the receiving ultrasonic waves as a result of coherent summation on the skin. The 3D images are visualized on a portable device 871 remote from the patient that enables real-time functioning. The interferometer 880 detects those maximum vibrational waves within the search area established by the sonographer or the doctor and assign xyz location to every vibrational point detected. All those points are combined together to create a heat grid map at that specific depth. The lasers are regulated to have a higher or lower penetration rate so it is possible to create another layer (another grid map) at a different depth but within the same investigational area mentioned above. Repeating the scanning process for several depths will provide the sonographer with different layers of the preferred area of investigation. At this point it is possible to simply interpolate the different layers in order to form a 3D image completely contactless.

The lens apparatus 831 is able to guide the two ultrasonic emitting beams 850. The lens apparatus 831 can also be used to guide the laser beams 850 in a specific area of the body.

Between two different laser pulses from the two laser sources is the moment where the maximum vibrational point is optically observed by the interferometer on the skin. That point is assigned specific xyz coordinates that will constitute a heat grid map. The laser beams may move, or be guided through the lens apparatus, on top of the search area established by the doctor or the sonographer and move according to specific patterns such as lawn-mower.

Additional real-time grid map construction can be built using filtering techniques, specifically using non-parametric filters such as particle filter techniques.

EXAMPLE 9: Simultaneous and synchronized Q-Switched pulsed lasers, colored camera, black and white camera, interferometer, GPS and 3-axis IMU.

FIG. 9 illustrates an exemplary system 900 configured to implement the present invention for 3D reconstruction of images with two excitation sources 930 and 940 configured to be implemented into the system working simultaneously and in synchronization with external sensors such as a colored camera 910 and a black and white camera 920, the 3-axis IMU (integrated in the structure 980), the GPS (integrated in the structure 980), or RTK GPS antenna, for real-time positioning and the interferometer 960. 951 simply represents the field of view of the lasers.

Images taken from a colored camera 910 and a black and white camera 920 are used to form stereo images and provide a 3D external shape of the patient 990. Patient's movement is compensated via real-time visual inertial odometry. Stereo images are used during the external reconstruction of the human shape providing a state of the art measured image. Knowing the position of the two cameras allows precise measurement of the patient 990. The 3-axis IMU used for real-time position and orientation integrated in the structure 980 is not visible but is present. Additionally a 3-axis IMU is integrated in the GPS (not visible but is present in the structure 980), or an RTK GPS antenna, in every Q-Switched laser 930 and 940, in the colored camera 910, in the black and white camera 930 and in the interferometer 960. Since a patient may move during the ultrasound session, the sonographer or the doctor can highlight specific points on the skin of the patient with a highlighter allowing the cameras to feature-detect those points and have a specific search area.

Two Q-switched pulsed lasers 930 and 940 emit an ultrasonic wave on top of the human body, or on the preferred part, that will propagate in the internal part of the body. Through a backscatter coherent summation of the return waves up to the skin of the patient it is possible to detect such displacement via an interferometric system 960. Lasers 930 and 940 operate simultaneously and in synchronization with the other external sensors.

In a hospital setting system 900 fits all around the patient and all the external sensors such as Q-Switched pulsed laser, colored camera, black and white camera, interferometer, GPS are present. All the sensors can be fixed while the platform 590 moves (translates) accordingly after the input of the doctor's or the sonographer's about the part to scan. Alternatively, the sensors remain fixed and the system 900 translates and rotates accordingly on top of the search area decided by the doctor by way of stepping motors.

The ultrasonic wave is emitted in the wavelength where the absorption is preferred by the human body and its internal tissues or organs in order to have an optimal response.

The interferometer 960 detects those maximum vibrational waves within the area the doctor's or the sonographer's decided to scan and assign xyz location to every point. All those points are combined together to create a heat grid map at that specific depth.

961 simply represents an interferometer observing the maximum vibration detected on the skin at a specific point. Returning waves on the skin outside of the field of view of the interferometer will only give a partial or incomplete reading. Therefore, it is important that the field of view of the two lasers 951 and the field of view of the interferometer overlap for optimal detection.

The lasers are regulated to have a higher or lower penetration rate so it is possible to create another layer (another grid map) at a different depth but within the same investigational area mentioned above. Repeating the scanning process for several depths will provide the sonographer with different layers of the preferred area of investigation. At this point it is possible to simply interpolate the different layers in order to form a 3D image completely contactless.

Between two different laser pulses from the two laser sources is the moment where the maximum vibrational point is optically observed by the interferometer on the skin. That point is assigned specific xyz coordinates that will constitute a heat grid map. The laser beams may move, or be guided through the lens apparatus, on top of the search area established by the doctor or the sonographer and move according to specific patterns such as lawn-mower.

Additional real-time grid map construction can be built using filtering techniques, specifically using non-parametric filters such as particle filter techniques.

EXAMPLE 10: Portable system.

In case of an exemplary handheld device as shown in FIG. 10, the system 1000 and the same sensors are used such as a colored camera 1010 and a black and white camera 1020, the 3-axis IMU 1090, GPS 1080, or RTK GPS antenna, for real-time positioning, the Q-switched lasers 1030 and 1040 and the interferometer 1070.

The 3-axis IMU 1090 is able to provide a precise orientation together with linear and angular velocities being able to detect the smallest movement in roll, pitch and yaw.

The 3-axis IMU 1090 integrates multi-axis, accelerometers and gyroscopes altogether to provide an object's orientation in space. Together the emitting laser beams 1050 and the external interferometer 1070 are able to detect the receiving ultrasonic waves as a result of coherent summation on the skin. The 3D images are visualized on a portable device 1081 remote from the patient that enables real-time functioning. The interferometer 1070 detects those maximum vibrational waves within the search area established by the sonographer or the doctor and assign xyz location to every vibrational point detected. All those points are combined together to create a heat grid map at that specific depth. The lasers are regulated to have a higher or lower penetration rate so it is possible to create another layer (another grid map) at a different depth but within the same investigational area mentioned above. Repeating the scanning process for several depths will provide the sonographer with different layers of the preferred area of investigation. At this point it is possible to simply interpolate the different layers in order to form a 3D image completely contactless.

Between two different laser pulses from the two laser sources is the moment where the maximum vibrational point is optically observed by the interferometer on the skin. That point is assigned specific xyz coordinates that will constitute a heat grid map. The laser beams may move, or be guided through the lens apparatus, on top of the search area established by the doctor or the sonographer and move according to specific patterns such as lawn-mower.

Additional real-time grid map construction can be built using filtering techniques, specifically using non-parametric filters such as particle filter techniques.

EXAMPLE 11: Simultaneous and synchronized Q-Switched pulsed laser, colored camera, black and white camera, interferometer, GPS and 3-axis IMU.

FIG. 11 illustrates an exemplary system 1100 configured to implement the present invention for 3D reconstruction of images with two excitation sources 1130 and 1140 configured to be implemented into the system working simultaneously and in synchronization together with the external sensors such as a colored camera 1110, a black and white camera 1120, the 3-axis IMU (integrated in the structure 1191), the GPS (integrated in the structure 1191), or RTK GPS antenna, for real-time positioning and the interferometer 1150. 1161 simply represents the field of view of the lasers.

Images taken from a colored camera 1110 and a black and white camera 1120 are used to form stereo images and provide a 3D external shape of the patient 1190. Patient's movement is compensated via real-time visual inertial odometry. Stereo images are used during the external reconstruction of the human shape providing a state of the art measured image. Knowing the position of the two cameras allows precise measurement of the patient 1190. The 3-axis IMU used for real-time position and orientation integrated in the structure 1191 is not visible but is present. Additionally a 3-axis IMU is integrated in the GPS (not visible but is present in the structure 1191), or an RTK GPS antenna, in every Q-Switched laser 1130 and 1140, in the colored camera 1110, in the black and white camera 1120 and in the interferometer 1150. Since a patient may move during the ultrasound session, the sonographer or the doctor can highlight specific points on the skin of the patient with a highlighter allowing the cameras to feature-detect those points and have a specific search area.

Two Q-switched pulsed lasers 1130 and 1140 emit an ultrasonic wave on top of the human body, or on the preferred part, that will propagate in the internal part of the body. Through a backscatter coherent summation of the return waves up to the skin of the patient it is possible to detect such displacement via an interferometric system 1150. Lasers 1130 and 1140 operate simultaneously and in synchronization with the other external sensors.

In a hospital setting system 1100 fits all around the patient and all the external sensors such as Q-Switched pulsed lasers, colored camera, black and white camera, interferometer, GPS are present. All the sensors can be fixed while the platform 1191 moves (translates) accordingly after the input of the doctor's or the sonographer's about the part to scan. Alternatively, the sensors remain fixed and the system 1100 translates and rotates accordingly on top of the search area decided by the doctor by way of stepping motors.

The ultrasonic wave is emitted in the wavelength where the absorption is preferred by the human body and its internal tissues or organs in order to have an optimal response.

The interferometer 1150 detects those maximum vibrational waves within the area the doctor's or the sonographer's decided to scan and assign xyz location to every point. All those points are combined together to create a heat grid map at that specific depth.

1180 simply represents an interferometer observing the maximum vibration detected on the skin at a specific point. Returning waves on the skin outside of the field of view of the interferometer will only give a partial or incomplete reading. Therefore, it is important that the field of view of the two lasers 1161 and the field of view of the interferometer overlap for optimal detection.

The lasers are regulated to have a higher or lower penetration rate so it is possible to create another layer (another grid map) at a different depth but within the same investigational area mentioned above. Repeating the scanning process for several depths will provide the sonographer with different layers of the preferred area of investigation. At this point it is possible to simply interpolate the different layers in order to form a 3D image completely contactless.

Between two different laser pulses from the two laser sources is the moment where the maximum vibrational point is optically observed by the interferometer on the skin. That point is assigned specific xyz coordinates that will constitute a heat grid map. The laser beams may move, or be guided through the lens apparatus, on top of the search area established by the doctor or the sonographer and move according to specific patterns such as lawn-mower.

Additional real-time grid map construction can be built using filtering techniques, specifically using non-parametric filters such as particle filter techniques.

EXAMPLE 12: Portable system.

In case of an exemplary handheld device as shown in FIG. 12, the system 1200 and the same sensors are used such as a colored camera 1210 and a black and white camera 1220, the 3-axis IMU 1290, GPS 1280, or RTK GPS antenna, for real-time positioning, the Q-switched lasers 1230 and 1240 and the interferometer 1270.

The 3-axis IMU 1290 is able to provide a precise orientation together with linear and angular velocities being able to detect the smallest movement in roll, pitch and yaw.

The 3-axis IMU 1290 integrates multi-axis, accelerometers and gyroscopes altogether to provide an object's orientation in space. Together the emitting laser beams 1250 and the external interferometer 1270 are able to detect the receiving ultrasonic waves as a result of coherent summation on the skin. The 3D images are visualized on a portable device 1281 remote from the patient that enables real-time functioning. The interferometer 1270 detects those maximum vibrational waves within the search area established by the sonographer or the doctor and assign xyz location to every vibrational point detected. All those points are combined together to create a heat grid map at that specific depth. The lasers are regulated to have a higher or lower penetration rate so it is possible to create another layer (another grid map) at a different depth but within the same investigational area mentioned above. Repeating the scanning process for several depths will provide the sonographer with different layers of the preferred area of investigation. At this point it is possible to simply interpolate the different layers in order to form a 3D image completely contactless.

Between two different laser pulses from the two laser sources is the moment where the maximum vibrational point is optically observed by the interferometer on the skin. That point is assigned specific xyz coordinates that will constitute a heat grid map. The laser beams may move, or be guided through the lens apparatus, on top of the search area established by the doctor or the sonographer and move according to specific patterns such as lawn-mower.

Additional real-time grid map construction can be built using filtering techniques, specifically using non-parametric filters such as particle filter techniques.

EXAMPLE 13: Flow chart explaining the post-processing procedure and image reconstruction for a single source Q-Switched pulsed laser, colored camera, black and white camera, interferometer, GPS and 3-axis IMU.

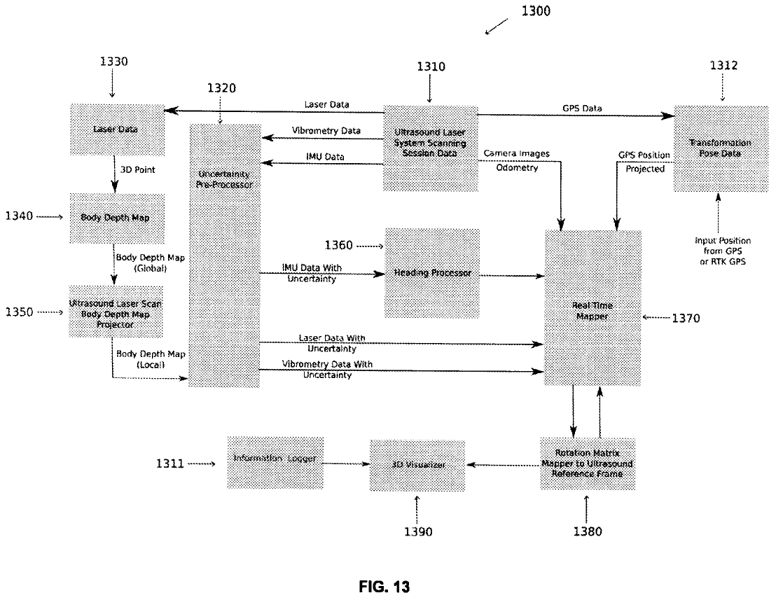

Referring to FIG. 13, a flow chart setting forth the most important steps for post-processing and generating laser images is provided. To start the post-processing loop and generate 2D/3D laser images (i.e. playing back an ultrasound session using a Robotic Platform such as ROS) related to the architecture shown in FIG. 1 or FIG. 3, the user needs to start from where all the data from the scanning session is collected, which is the scanning session data 1310. The ultrasound laser system scanning session data 1310 is the main container of the recorded ultrasound session. Such data are, for example, displacement, position, velocity and acceleration of every sensor and even monitoring the movements of the stepper motors. The presence of the GPS makes sure that there is an absolute reference frame from which all the other local reference frames given from the other sensors relate to while the system translates or roto-translate around the patient.

All necessary data coming from laser sources, external sensors such as color camera, black and white camera, 3-axis IMU, interferometer and a GPS, or an RTK GPS antenna system, 1312 are characterized by an intrinsic uncertainty in the measurements.

Therefore, given the uncertainty values of the measurement typically indicated by the manufacturer of the device, the values are all pre-processed inside a block called the pre-processor of uncertainty 1320. All the outputs of the pre-processor of uncertainty are fully carrying the uncertainty values. Some output from the pre-processor of uncertainty 1320, such as the laser data and the vibrometry data, don't need to be further processed and can be implemented into the real-time mapper system 1370.

Other measurements, such as 3-axis IMU data with uncertainty of the other sensors, need further processing as it is required to transform all the necessary orientation, linear and angular velocities into a specific direction. This is even more important in the case of a hand-held device or a head wearable device. This 3-axis IMU data with uncertainty needs to be transformed therefore into heading via a heading processor 1360. After that these 3-axis IMU values can be processed in the real-time mapper system 1370. An additional sensor such as a GPS, or an RTK GPS antenna system, represented by the block 1312 can be implemented to set the absolute reference frame.

In order to make sure that the images are properly reconstructed, a proper transformation via rotation matrix 1380 from Real-Time Mapper to Ultrasound Reference frame may be required as shown in FIG. 13. Displacement of laser images may allow for feature detection and therefore point-to-point registration allowing for a better image formation. In addition and at later steps, outliers may also be neglected for a better performance of the calculation.

Results are shown into a 3D visualizer 1390. The post-processing system related to the architecture shown in FIG. 1 or FIG. 3 is equipped with an information logger 1311 able to record every input/output of each sensor in case the user would like to change or tune some of the sensors parameters to carry additional studies post ultrasound exam or perform different simulation and experimenting specific scenarios by modifying specific parameters. Therefore, using the Robotic Platform, makes it possible to playback the ultrasound session for that specific patient given the recording of the movement of all the external sensors and their local relative reference frames with relation to the absolute reference frame provided by the GPS.

EXAMPLE 14: Flow chart explaining the post-processing procedure and image reconstruction for a single source Q-Switched pulsed laser, colored camera, black and white camera, interferometer, GPS and 3-axis IMU.

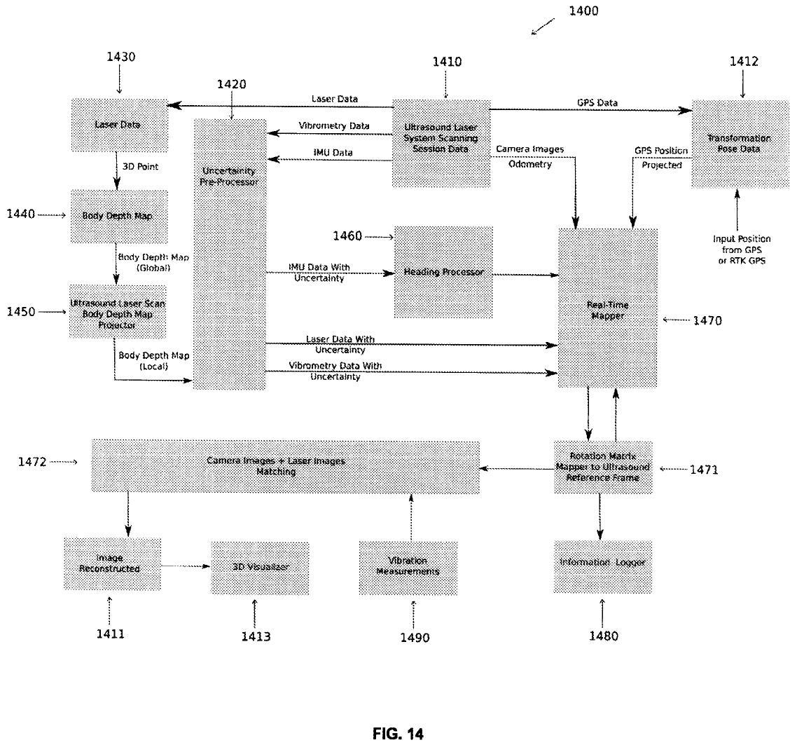

Referring to FIG. 14, a flow chart setting forth the most important steps for post-processing and generating laser images is provided. To start the post-processing loop and generate 2D/3D laser images (i.e. playing back an ultrasound session using a Robotic Platform such as ROS) related to the architecture shown in FIG. 1 or FIG. 3, the user needs to start from where all the data from the scanning session is collected, which is the scanning session data 1410. The ultrasound laser system scanning session data 1410 is the main container of the recorded ultrasound session. Such data are, for example, displacement, position, velocity and acceleration of every sensor and even monitoring the movements of the stepper motors. The presence of the GPS makes sure that there is an absolute reference frame from which all the other local reference frames given from the other sensors relate to while the system translates or roto-translate around the patient.

All necessary data coming from laser sources, external sensors such as color camera, black and white camera, 3-axis IMU, interferometer and a GPS, or an RTK GPS antenna system, 1412 are characterized by an intrinsic uncertainty in the measurements.

Therefore, given the uncertainty values of the measurement typically indicated by the manufacturer of the device, the values are all pre-processed inside a block called the pre-processor of uncertainty 1420. All the outputs of the pre-processor of uncertainty are fully carrying the uncertainty values. Some output such as the laser data and the vibrometry data don't need to be further processed and can be implemented into the real-time mapper system 1470.

Other measurements such as 3-axis IMU data with uncertainty of the other sensors, need further processing as it is required to transform all the necessary orientation, linear and angular velocities into a specific direction. This is even more important in the case of a hand-held device or a head wearable device. This 3-axis IMU data with uncertainty needs to be transformed therefore into heading via a heading processor 1460. After that these 3-axis IMU values can be processed in the real-time mapper system 1470. An additional sensor such as a GPS, or an RTK GPS antenna system, represented by the block 1412 can be implemented to set the absolute reference frame.

In order to make sure that the images are properly reconstructed, a proper transformation via rotation matrix 1471 from Real-Time Mapper to Ultrasound Reference frame may be required as shown in FIG. 14. Displacement of laser images may allow for feature detection and therefore point-to-point registration allowing for a better image formation. In addition and at later steps, outliers may also be neglected for a better performance of the calculation. The additional block 1472 shows that images properly processed may be subjected to an additional matching step where outside stereo images taken using the cameras overlap with the vibrometric images (grid heat map) optically detected at that depth using the interferometer. So any vibrational point outside of the search area can be considered an outlier and, therefore, neglected.

Results are shown into a 3D visualizer 1413. The post-processing system related to the architecture shown in FIG. 1 or FIG. 3 is equipped with an information logger 1480 able to record every input/output of each sensor in case the user would like to change or tune some of the sensors parameters to carry additional studies post ultrasound exam or perform different simulation and experimenting specific scenarios by modifying specific parameters. Therefore, using the Robotic Platform, makes it possible to playback the ultrasound session for that specific patient given the recording of the movement of all the external sensors and their local relative reference frames with relation to the absolute reference frame provided by the GPS.

EXAMPLE 15: Flow chart explaining the post-processing procedure and image reconstruction for a two source Q-Switched pulsed laser working simultaneously and in synchronization, colored camera, black and white camera, interferometer, GPS and 3-axis IMU.

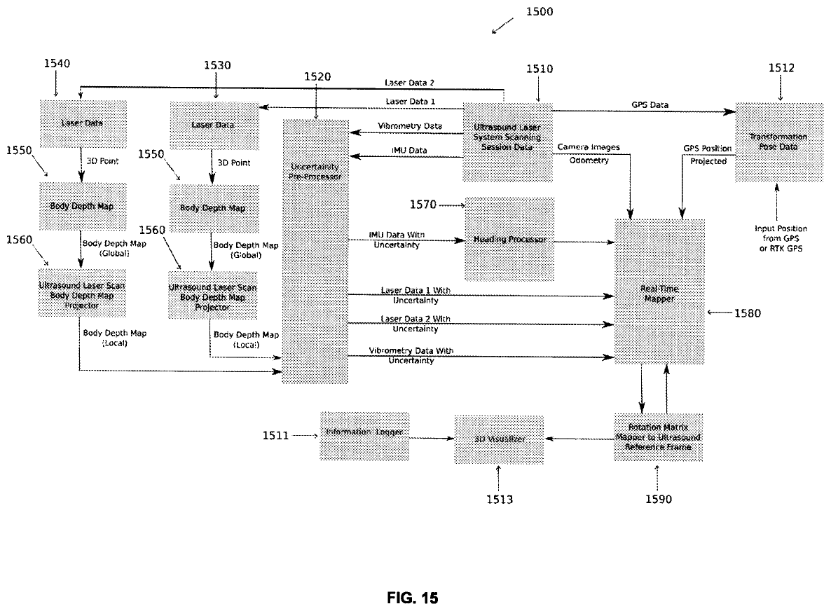

Referring to FIG. 15, a flow chart setting forth the most important steps for post-processing and generating laser images is provided. To start the post-processing loop and generate 2D/3D laser images (i.e. playing back an ultrasound session using a Robotic Platform such as ROS) related to the architecture shown in FIG. 5, FIG. 7, FIG. 9 or FIG. 11, the user needs to start from where all the data from the scanning session is collected, which is the scanning session data 1510. The ultrasound laser system scanning session data 1510 is the main container of the recorded ultrasound session. Such data are, for example, displacement, position, velocity and acceleration of every sensor and even monitoring the movements of the stepper motors. The presence of the GPS makes sure that there is an absolute reference frame from which all the other local reference frames given from the other sensors relate to while the system translates or roto-translate around the patient.

All necessary data coming from laser sources, external sensors such as color camera, black and white camera, 3-axis IMU, interferometer and a GPS, or an RTK GPS antenna system, 1512 are characterized by an intrinsic uncertainty in the measurements.

Therefore, given the uncertainty values of the measurement typically indicated by the manufacturer of the device, the values are all pre-processed inside a block called the pre-processor of uncertainty 1520. All the outputs of the pre-processor of uncertainty are fully carrying the uncertainty values. Some output from the pre-processor of uncertainty 1520 such as the laser sources and the vibrometry data don't need to be further processed and can be implemented into the real-time mapper system 1580.

Other measurements such as 3-axis IMU data with uncertainty of the other sensors, need further processing as it is required to transform all the necessary orientation, linear and angular velocities into a specific direction. This is even more important in the case of a hand-held device or a head wearable device. This 3-axis IMU data with uncertainty needs to be transformed therefore into heading via a heading processor 1570. After that these 3-axis IMU values can be processed in the real-time mapper system 1580. An additional sensor such as a GPS, or an RTK GPS antenna system, represented by the block 1512 can be implemented to set the absolute reference frame.

In order to make sure that the images are properly reconstructed, a proper transformation via rotation matrix 1590 from Real-Time Mapper to Ultrasound Reference frame may be required as shown in FIG. 15. In addition and at later steps, outliers may also be neglected for a better performance of the calculation.

Results are shown into a 3D visualizer 1513. The post-processing system related to the architecture shown in FIG. 5, FIG. 7, FIG. 9 or FIG. 11 is equipped with an information logger 1511 able to record every input/output of each sensor in case the user would like to change or tune some of the sensors parameters to carry additional studies post ultrasound exam or perform different simulation and experimenting specific scenarios by modifying specific parameters. Therefore, using the Robotic Platform, makes it possible to playback the ultrasound session for that specific patient given the recording of the movement of all the external sensors and their local relative reference frames with relation to the absolute reference frame provided by the GPS.

EXAMPLE 16: Flow chart explaining the post-processing procedure and image reconstruction for a two source Q-Switched pulsed laser working simultaneously and in synchronization, colored camera, black and white camera, interferometer, GPS and 3-axis IMU.

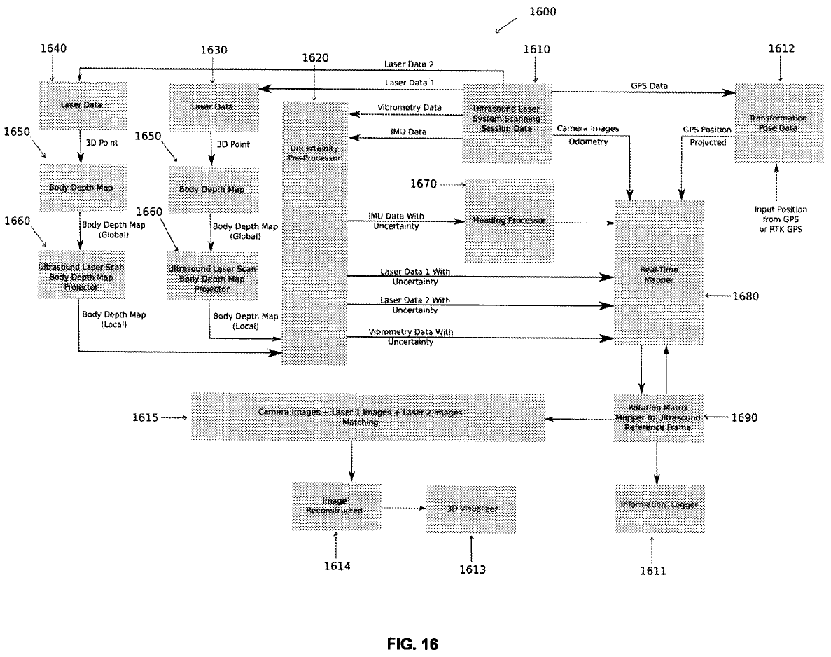

Referring to FIG. 16, a flow chart setting forth the most important steps for post-processing and generating laser images is provided. To start the post-processing loop and generate 2D/3D laser images (i.e. playing back an ultrasound session using a Robotic Platform such as ROS) related to the architecture shown in FIG. 5, FIG. 7, FIG. 9 or FIG. 11, the user needs to start from where all the data from the scanning session is collected, which is the scanning session data 1610. The ultrasound laser system scanning session data 1610 is the main container of the recorded ultrasound session. Such data are, for example, displacement, position, velocity and acceleration of every sensor and even monitoring the movements of the stepper motors. The presence of the GPS makes sure that there is an absolute reference frame from which all the other local reference frames given from the other sensors relate to while the system translates or roto-translate around the patient.

All necessary data coming from laser sources, external sensors such as color camera, black and white camera, 3-axis IMU, interferometer and a GPS, or an RTK GPS antenna system, 1612 are characterized by an intrinsic uncertainty in the measurements.

Therefore, given the uncertainty values of the measurement typically indicated by the manufacturer of the device, the values are all pre-processed inside a block called the pre-processor of uncertainty 1620. All the outputs of the pre-processor of uncertainty are fully carrying the uncertainty values. Some output from the pre-processor of uncertainty 1620 such as the laser sources and the vibrometry data don't need to be further processed and can be implemented into the real-time mapper system 1680.

Other measurements such as 3-axis IMU data with uncertainty of the other sensors, need further processing as it is required to transform all the necessary orientation, linear and angular velocities into a specific direction. This is even more important in the case of a hand-held device or a head wearable device. The 3-axis IMU data with uncertainty needs to be transformed therefore into heading via a heading processor 1670. After that these 3-axis IMU values can be processed in the real-time mapper system 1680. An additional sensor such as a GPS, or an RTK GPS antenna system, represented by the block 1612 can be implemented to set the absolute reference frame.

In order to make sure that the images are properly reconstructed, a proper transformation via rotation matrix 1690 from Real-Time Mapper to Ultrasound Reference frame may be required as shown in FIG. 14. Displacement of laser images may allow for feature detection and therefore point-to-point registration allowing for a better image formation. In addition and at later steps, outliers may also be neglected for a better performance of the calculation. The additional block 1615 shows that images properly processed may be subjected to an additional matching step where outside stereo images taken using the cameras overlap with the vibrometric images (grid heat map) optically detected at that depth using the interferometer. So any vibrational point outside of the search area can be considered an outlier and, therefore, neglected.

Results of the exam are shown into a 3D visualizer 1613. The post-processing system related to the architecture shown in FIG. 5, FIG. 7, FIG. 9 or FIG. 11 is equipped with an information logger 1611 able to record every input/output of each sensor in case the user would like to change or tune some of the sensors parameters to carry additional studies post ultrasound exam or perform different simulation and experimenting specific scenarios by modifying specific parameters. Therefore, using the Robotic Platform, makes it possible to playback the ultrasound session for that specific patient given the recording of the movement of all the external sensors and their local relative reference frames with relation to the absolute reference frame provided by the GPS.

EXAMPLE 17: Flow chart explaining the real-time processing procedure and image reconstruction for a single source Q-Switched pulsed laser, colored camera, black and white camera, interferometer, GPS and 3-axis IMU.

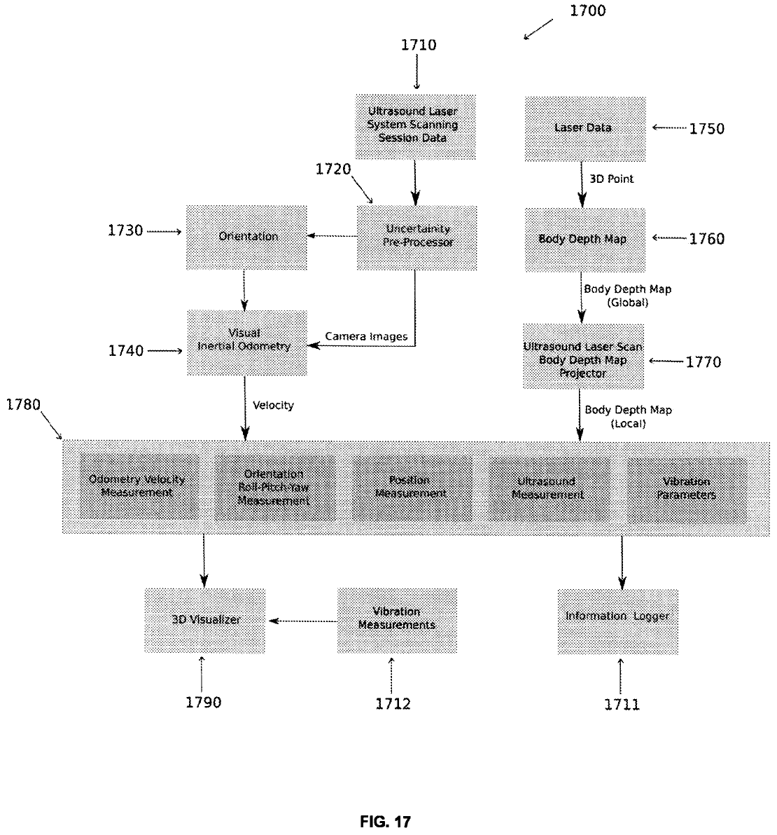

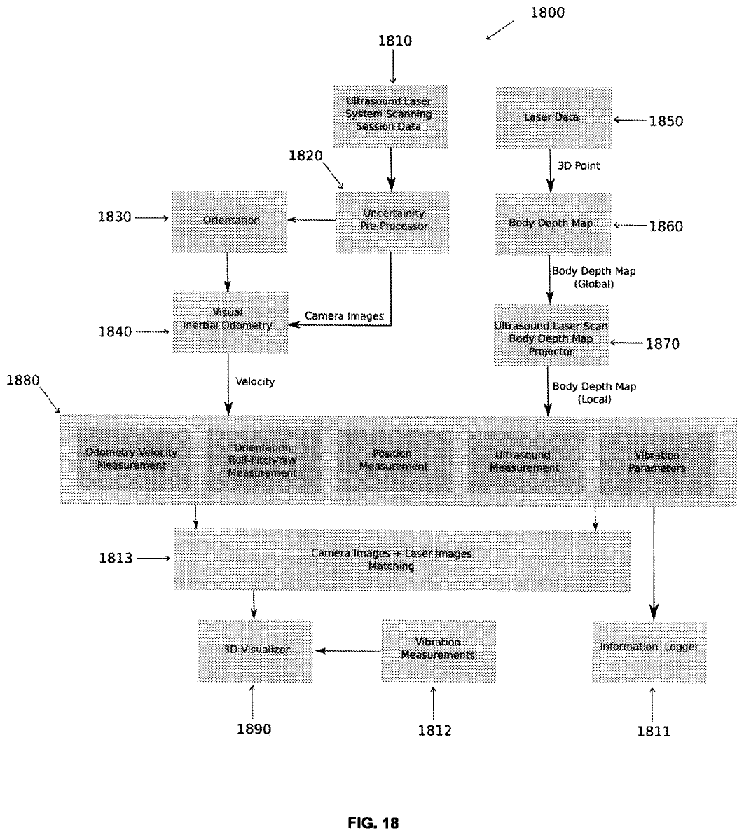

Referring to FIG. 17, a flow chart setting forth the most important steps for generating laser images in real-time is provided. To start the real-time processing loop 1700 and generate 2D/3D laser images related to the architecture shown in FIG. 1 or FIG. 3 one photoacoustic laser source is directed toward the patient.

Laser source 1750 emits ultrasonic disturbances towards the patient, external sensors are used to precisely reconstruct the images, and are characterized by an intrinsic uncertainty in the measurements.

Therefore, given the uncertainty values of the measurement typically indicated by the manufacturer of the device, the values are all pre-processed inside a block called the pre-processor of uncertainty 1720. All the outputs of the pre-processor of uncertainty are fully carrying the uncertainty values. The uncertainty values are therefore passed to the orientation block 1730, provided by the 3-axis IMU, and the images taken by the cameras resulting in the visual inertial odometry block 1740. Camera images may be used to estimate the velocity of the system as it moves, calculating the displacement between sequential images and their direction and orientation in order to keep the search box area established by the doctor or the sonographer always in the field of view.

Outputs from the visual inertial odometry, 3-axis IMU and orientation are then passed to the filtering system 1780 composed of five main blocks: the odometry velocity measurement, the orientation roll-pitch-yaw measurement, position measurement, ultrasound measurement and vibrometry measurement. In order to provide 2D/3D images the filtering system uses the technique of particle filtering. Real-time measurements coming from all the synchronized sensors are used to build the layer of the heat grid map at that specific depth given the penetration rate of the laser. In order to predict what the next measurement will be (i.e. the next depth measurement) historical and current vibrational data points are used to predict future vibrational points, providing insights into what the next depth measurement might be.

The representation of the five blocks and therefore the vibrational layer (heat grid map) at a specific depth are therefore visualized in a 3D visualizer monitor 1790. The real-time system related to the architecture shown in FIG. 1 or FIG. 3 is equipped with an information logger 1711 able to record every input/output of each sensor in case the user would like to change or tune some of the sensors parameters to carry additional studies post ultrasound exam or perform different simulation and experimenting specific scenarios by modifying specific parameters. Therefore, using the Robotic Platform, makes it possible to show real-time images for that specific patient given the movement of all the external sensors and their local relative reference frames with relation to the absolute reference frame provided by the GPS.

By way of example related to FIG. 17: Q-Switched laser A sends a pulse a time 0 at position 0 so the first wave can penetrate the surface of the skin and arrive at the preferred depth. The system translates within the investigational area decided by the doctor and the same Q-Switched laser A sends another pulse, for example, 10 nanoseconds later a time 1 at position 1 so the second wave can penetrate the surface of the skin and arrive at the preferred depth. The interval between the two pulses is when the maximum surface wave vibration is detected from the external interferometer. So in real-time, the system translates (or roto-translates) at extremely low speed within the search area defined by the doctor alternating pulses from the same laser. Maximum vibrational surface points are located by the interferometer and then translated into a proper grid heat map after compensating for rotation and scale. The particle filter algorithm can be used to predict what the next maximum vibrational point can be as it is able to find patterns in historical and current data to extend them into future predictions. In this way real-time is possible.

After patient A finishes an ultrasound session, the doctor has the 2D/3D heat grid map. If patient A comes back a few months later the doctor may upload the previous vibrational map as an input 1712 on the 3D visualizer 1790 and at the same time perform a new scan. At the end of the ultrasound session, the doctor may compare the two vibrational maps to see if any changes inside the tissue occurred and act accordingly.

EXAMPLE 18: Flow chart explaining the real-time processing procedure and image reconstruction for a single source Q-Switched pulsed laser, colored camera, black and white camera, interferometer, GPS and 3-axis IMU.

Referring to FIG. 18, a flow chart setting forth the most important steps for generating laser images in real-time is provided. To start the real-time processing loop 1800 and generate 2D/3D laser images related to the architecture shown in FIG. 1 or FIG. 3 one photoacoustic laser source is directed toward the patient.

Laser source 1850 emits ultrasonic disturbances towards the patient, external sensors are used to precisely reconstruct the images, and are characterized by an intrinsic uncertainty in the measurements.

Therefore, given the uncertainty values of the measurement typically indicated by the manufacturer of the device, the values are all pre-processed inside a block called the pre-processor of uncertainty 1820. All the outputs of the pre-processor of uncertainty are fully carrying the uncertainty values. The uncertainty values are therefore passed to the orientation block 1830, provided by the 3-axis IMU, and the images taken by the cameras resulting in the visual inertial odometry block 1840. Camera images may be used to estimate the velocity of the system as it moves, calculating the displacement between sequential images and their direction and orientation in order to keep the search box area established by the doctor or the sonographer always in the field of view.

Outputs from the visual inertial odometry, 3-axis IMU and orientation are then passed to the filtering system 1880 composed of five main blocks: the odometry velocity measurement, the orientation roll-pitch-yaw measurement, position measurement, ultrasound measurement and vibrometry measurement. In order to provide 2D/3D images the filtering system uses the technique of particle filtering. Real-time measurements coming from all the synchronized sensors are used to build the layer of the heat grid map at that specific depth given the penetration rate of the laser. In order to predict what the next measurement will be (i.e. the next depth measurement) historical and current vibrational data points are used to predict future vibrational points, providing insights into what the next depth measurement might be.

The representation of the five blocks and therefore the vibrational layer (heat grid map) at a specific depth are therefore visualized in a 3D visualizer monitor 1890. The real-time system related to the architecture shown in FIG. 1 or FIG. 3 is equipped with an information logger 1811 able to record every input/output of each sensor in case the user would like to change or tune some of the sensors parameters to carry additional studies post ultrasound exam or perform different simulation and experimenting specific scenarios by modifying specific parameters. Therefore, using the Robotic Platform, makes it possible to show real-time images for that specific patient given the movement of all the external sensors and their local relative reference frames with relation to the absolute reference frame provided by the GPS.

By way of example related to FIG. 17: Q-Switched laser A sends a pulse a time 0 at position 0 so the first wave can penetrate the surface of the skin and arrive at the preferred depth. The system translates within the investigational area decided by the doctor and the same Q-Switched laser A sends another pulse, for example, 10 nanoseconds later a time 1 at position 1 so the second wave can penetrate the surface of the skin and arrive at the preferred depth. The interval between the two pulses is when the maximum surface wave vibration is detected from the external interferometer. So in real-time, the system translates (or roto-translates) at extremely low speed within the search area defined by the doctor alternating pulses from the same laser. Maximum vibrational surface points are located by the interferometer and then translated into a proper grid heat map after compensating for rotation and scale. The particle filter algorithm can be used to predict what the next maximum vibrational point can be as it is able to find patterns in historical and current data to extend them into future predictions. In this way real-time is possible.

The additional block 1813 shows that images properly processed may be subjected to an additional matching step where outside stereo images taken using the cameras overlap with the vibrometric images (grid heat map) optically detected at that depth using the interferometer. So any vibrational point outside of the search area can be considered an outlier and, therefore, neglected in real-time.

After patient A finishes an ultrasound session, the doctor has the 2D/3D heat grid map. If patient A comes back a few months later the doctor may upload the previous vibrational map as an input 1812 on the 3D visualizer 1790 and at the same time perform a new scan. At the end of the ultrasound session, the doctor may compare the two vibrational maps to see if any changes inside the tissue occurred and act accordingly.

EXAMPLE 19: flow chart explaining the real-time processing procedure and image reconstruction for a two source Q-Switched pulsed laser working simultaneously and in synchronization, colored camera, black and white camera, interferometer, GPS and 3-axis IMU.

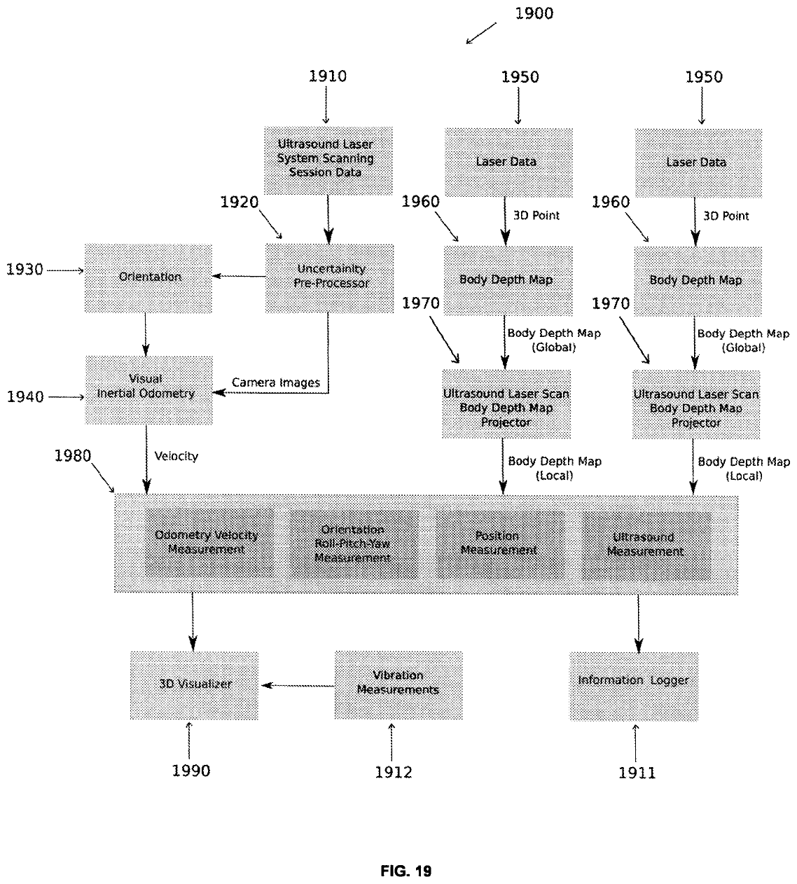

Referring to FIG. 19, a flow chart setting forth the most important steps for generating laser images in real-time is provided. To start the real-time processing loop 1900 and generate 2D/3D laser images related to the architecture shown in FIG. 5, FIG. 7, FIG. 9 or FIG. 11 two photoacoustics laser sources are directed toward the patient.

Laser sources 1950 emit ultrasonic disturbances towards the patient, external sensors are used to precisely reconstruct the images, and are characterized by an intrinsic uncertainty in the measurements.

Therefore, given the uncertainty values of the measurement typically indicated by the manufacturer of the device, the values are all pre-processed inside a block called the pre-processor of uncertainty 1920. All the outputs of the pre-processor of uncertainty are fully carrying the uncertainty values. The uncertainty values are therefore passed to the orientation block 1930, provided by the 3-axis IMU, and the images taken by the camera resulting in the visual inertial odometry block 1940. Camera images may be used to estimate the velocity of the system as it moves, calculating the displacement between sequential images and their direction and orientation in order to keep the search box area established by the doctor or the sonographer always in the field of view.

Outputs from the visual inertial odometry, 3-axis IMU and orientation are then passed to the filtering system 1980 composed of four main blocks: the odometry velocity measurement block, the orientation roll-pitch-yaw measurement, position measurement and ultrasound measurement. In order to provide 2D/3D images the filtering system uses the technique of particle filtering. Real-time measurements coming from all the synchronized sensors are used to build the layer of the heat grid map at that specific depth given the penetration rate of the laser. In order to predict what the next measurement will be (i.e. the next depth measurement) historical and current vibrational data points are used to predict future vibrational points, providing insights into what the next depth measurement might be.

The representation of the four blocks and therefore the vibrational layer (heat grid map) at a specific depth are therefore visualized in a 3D visualizer monitor 1990. The real-time system related to the architecture shown in FIG. 5, FIG. 7, FIG. 9 or FIG. 11 is equipped with an information logger 1911 able to record every input/output of each sensor in case the user would like to change or tune some of the sensors parameters to carry additional studies post ultrasound exam or perform different simulation and given the movement of all the external sensors and their local relative reference frames with relation to the absolute reference frame provided by the GPS.

By way of example: Q-Switched laser A sends a pulse a time 0 at position 0 so the first wave can penetrate the surface of the skin and arrive at the preferred depth. Q-Switched laser B sends a pulse, for example, 10 nanoseconds later a time 1 at position 1 so the second wave can penetrate the surface of the skin and arrive at the preferred depth. The interval between the two pulses from the two different lasers is when the maximum surface wave vibration is detected from the external interferometer. So in real-time, the system translates (or roto-translates) at extremely low speed within the search area defined by the doctor while it performs the scan alternating the two pulses from the two different lasers. Maximum vibrational surface points are located by the interferometer and then translated into a proper grid map after compensating for rotation and scale. The particle filter algorithm can be used to predict what the next maximum vibrational point can be as it is able to find patterns in historical and current data to extend them into future predictions. In this way real-time is possible.

After patient A finishes an ultrasound session, the doctor has the 2D/3D heat grid map. If patient A comes back a few months later the doctor may upload the previous vibrational map as an input 1912 on the 3D visualizer 1990 and at the same time perform a new scan. At the end of the ultrasound session, the doctor may compare the two vibrational maps to see if any changes inside the tissue occurred and act accordingly.

EXAMPLE 20: flow chart explaining the real-time processing procedure and image reconstruction for a two source Q-Switched pulsed laser working simultaneously and in synchronization, colored camera, black and white camera, interferometer, GPS and 3-axis IMU.

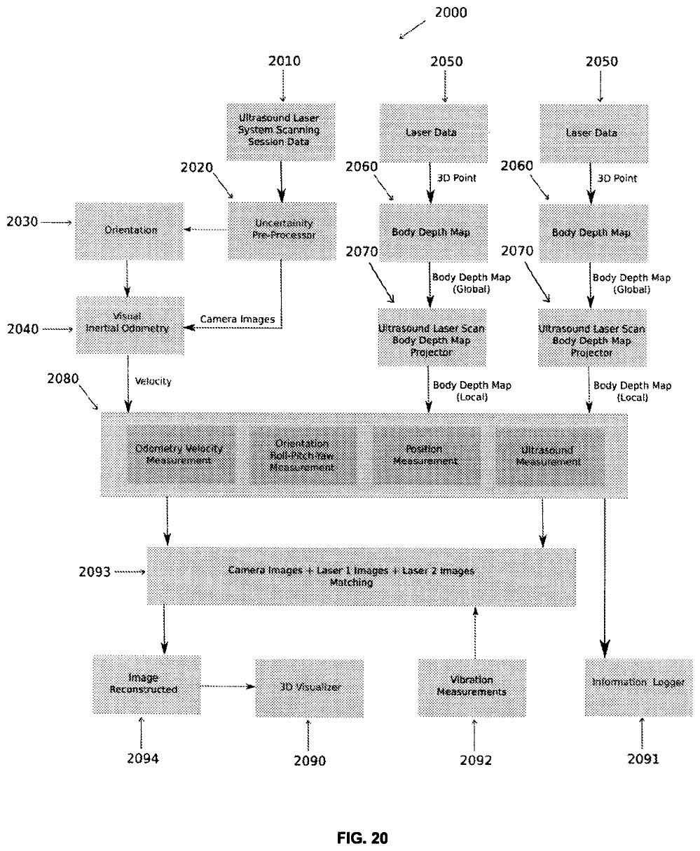

Referring to FIG. 20, a flow chart setting forth the most important steps for generating laser images in real-time is provided. To start the real-time processing loop 2000 and generate 2D/3D laser images related to the architecture shown in FIG. 5, FIG. 7, FIG. 9 or FIG. 11 two photoacoustics laser sources are directed toward the patient.

Laser sources 2050 emit ultrasonic disturbances towards the patient, external sensors are used to precisely reconstruct the images, and are characterized by an intrinsic uncertainty in the measurements.

Therefore, given the uncertainty values of the measurement typically indicated by the manufacturer of the device, the values are all pre-processed inside a block called the pre-processor of uncertainty 2020. All the outputs of the pre-processor of uncertainty are fully carrying the uncertainty values. The uncertainty values are therefore passed to the orientation block 2030, provided by the 3-axis IMU, and the images taken by the camera resulting in the visual inertial odometry block 2040. Camera images may be used to estimate the velocity of the system as it moves, calculating the displacement between sequential images and their direction and orientation in order to keep the search box area established by the doctor or the sonographer always in the field of view.

Outputs from the visual inertial odometry, 3-axis IMU and orientation are then passed to the filtering system 2080 composed of four main blocks: the odometry velocity measurement block, the orientation roll-pitch-yaw measurement, position measurement and ultrasound measurement. In order to provide 2D/3D images the filtering system uses the technique of particle filtering. Real-time measurements coming from all the synchronized sensors are used to build the layer of the heat grid map at that specific depth given the penetration rate of the laser. In order to predict what the next measurement will be (i.e. the next depth measurement) historical and current vibrational data points are used to predict future vibrational points, providing insights into what the next depth measurement might be.

The representation of the four blocks and therefore the vibrational layer (heat grid map) at a specific depth are therefore visualized in a 3D visualizer monitor 2090. The real-time system related to the architecture shown in FIG. 5, FIG. 7, FIG. 9 or FIG. 11 is equipped with an information logger 2091 able to record every input/output of each sensor in case the user would like to change or tune some of the sensors parameters to carry additional studies post ultrasound exam or perform different simulation and experimenting specific scenarios by modifying specific parameters. Therefore, using the Robotic Platform, makes it possible to show real-time images for that specific patient given the movement of all the external sensors and their local relative reference frames with relation to the absolute reference frame provided by the GPS.

By way of example: Q-Switched laser A sends a pulse a time 0 at position 0 so the first wave can penetrate the surface of the skin and arrive at the preferred depth. Q-Switched laser B sends a pulse, for example, 10 nanoseconds later a time 1 at position 1 so the second wave can penetrate the surface of the skin and arrive at the preferred depth. The interval between the two pulses from the two different lasers is when the maximum surface wave vibration is detected from the external interferometer. So in real-time, the system translates (or roto-translates) at extremely low speed within the search area defined by the doctor while it performs the scan alternating the two pulses from the two different lasers. Maximum vibrational surface points are located by the interferometer and then translated into a proper grid map after compensating for rotation and scale. The particle filter algorithm can be used to predict what the next maximum vibrational point can be as it is able to find patterns in historical and current data to extend them into future predictions. In this way real-time is possible.

The additional block 2093 shows that images properly processed may be subjected to an additional matching step where outside stereo images taken using the cameras overlap with the vibrometric images (grid heat map) optically detected at that depth using the interferometer. So any vibrational point outside of the search area can be considered an outlier and, therefore, neglected in real-time.

After patient A finishes an ultrasound session, the doctor has the 2D/3D heat grid map. If patient A comes back a few months later the doctor may upload the previous vibrational map as an input 2092 on the 3D matching block 2093 and at the same time perform a new scan. At the end of the ultrasound session, the doctor may compare the two vibrational maps to see if any changes inside the tissue occurred and act accordingly.

EXAMPLE 21: flow chart explaining the real-time processing procedure and image reconstruction for a two source Q-Switched pulsed laser working simultaneously and in synchronization, colored camera, black and white camera, interferometer, GPS and 3-axis IMU.

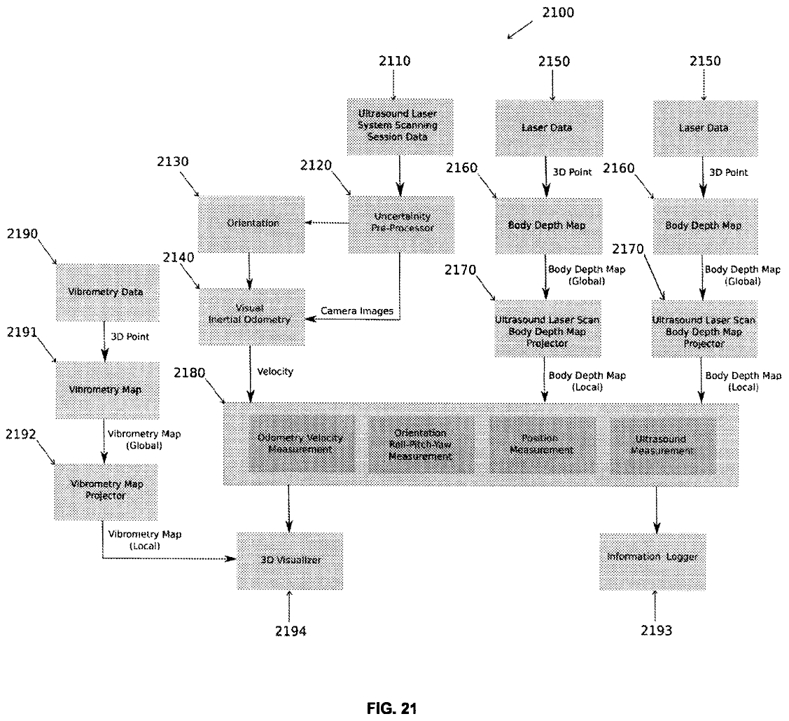

Referring to FIG. 21, a flow chart setting forth the most important steps for generating laser images in real-time is provided. To start the real-time processing loop 2100 and generate 2D/3D laser images related to the architecture shown in FIG. 5, FIG. 7, FIG. 9 or FIG. 11 two photoacoustics laser sources are directed toward the patient.

Laser sources 2150 emit ultrasonic disturbances towards the patient, external sensors are used to precisely reconstruct the images, and are characterized by an intrinsic uncertainty in the measurements.

Therefore, given the uncertainty values of the measurement typically indicated by the manufacturer of the device, the values are all pre-processed inside a block called the pre-processor of uncertainty 2020. All the outputs of the pre-processor of uncertainty are fully carrying the uncertainty values. The uncertainty values are therefore passed to the orientation block 2130, provided by the 3-axis IMU, and the images taken by the camera resulting in the visual inertial odometry block 2140. Camera images may be used to estimate the velocity of the system as it moves, calculating the displacement between sequential images and their direction and orientation in order to keep the search box area established by the doctor or the sonographer always in the field of view.

Outputs from the visual inertial odometry, 3-axis IMU and orientation are then passed to the filtering system 2180 composed of four main blocks: the odometry velocity measurement block, the orientation roll-pitch-yaw measurement, position measurement and ultrasound measurement. In order to provide 2D/3D images the filtering system uses the technique of particle filtering. Real-time measurements coming from all the synchronized sensors are used to build the layer of the heat grid map at that specific depth given the penetration rate of the laser. In order to predict what the next measurement will be (i.e. the next depth measurement) historical and current vibrational data points are used to predict future vibrational points, providing insights into what the next depth measurement might be.

The representation of the four blocks and therefore the vibrational layer (heat grid map) at a specific depth are therefore visualized in a 3D visualizer monitor 2194. The real-time system related to the architecture shown in FIG. 5, FIG. 7, FIG. 9 or FIG. 11 is equipped with an information logger 2193 able to record every input/output of each sensor in case the user would like to change or tune some of the sensors parameters to carry additional studies post ultrasound exam or perform different simulation and given the movement of all the external sensors and their local relative reference frames with relation to the absolute reference frame provided by the GPS.

By way of example: Q-Switched laser A sends a pulse a time 0 at position 0 so the first wave can penetrate the surface of the skin and arrive at the preferred depth. Q-Switched laser B sends a pulse, for example, 10 nanoseconds later a time 1 at position 1 so the second wave can penetrate the surface of the skin and arrive at the preferred depth. The interval between the two pulses from the two different lasers is when the maximum surface wave vibration is detected from the external interferometer. So in real-time, the system translates (or roto-translates) at extremely low speed within the search area defined by the doctor while it performs the scan alternating the two pulses from the two different lasers. Maximum vibrational surface points are located by the interferometer and then translated into a proper grid map after compensating for rotation and scale. The particle filter algorithm can be used to predict what the next maximum vibrational point can be as it is able to find patterns in historical and current data to extend them into future predictions. In this way real-time is possible.

After patient A finishes an ultrasound session, the doctor has the 2D/3D heat grid map. If patient A comes back a few months later the doctor may upload the previous vibrational map as an input 2190 on the 3D visualizer block 2194 and at the same time perform a new scan. At the end of the ultrasound session, the doctor may compare the two vibrational maps to see if any changes inside the tissue occurred and act accordingly. The present case is just a variant of example 20.

EXAMPLE 22: flow chart explaining the real-time processing procedure and image reconstruction for a two source Q-Switched pulsed laser working simultaneously and in synchronization, colored camera, black and white camera, interferometer, GPS and 3-axis IMU.

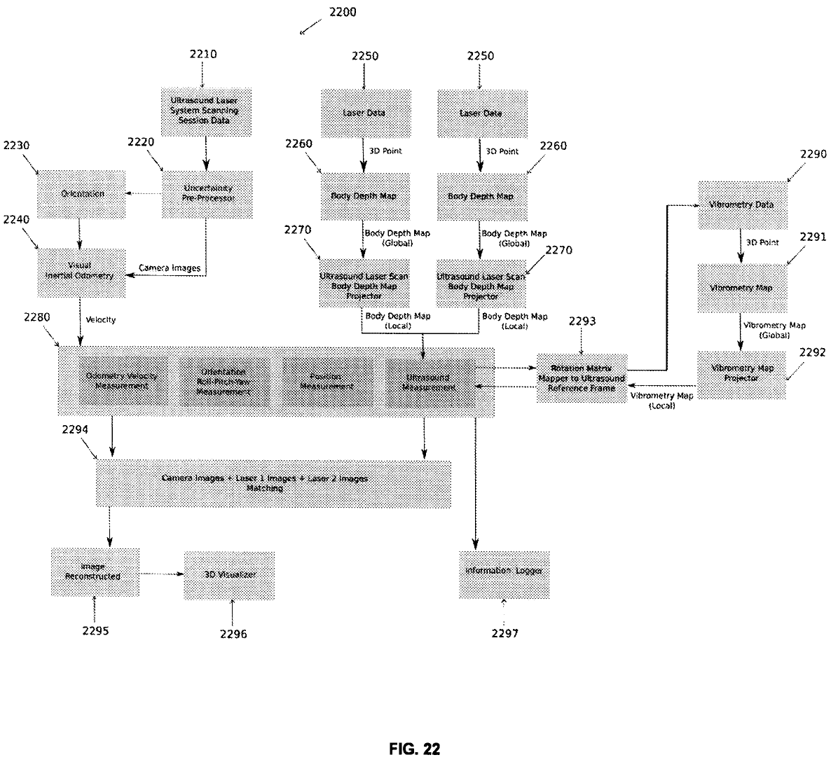

Referring to FIG. 22, a flow chart setting forth the most important steps for generating laser images in real-time is provided. To start the real-time processing loop 2200 and generate 2D/3D laser images related to the architecture shown in FIG. 5, FIG. 7, FIG. 9 or FIG. 11 two photoacoustics laser sources are directed toward the patient.

Laser sources 2250 emit ultrasonic disturbances towards the patient, external sensors are used to precisely reconstruct the images, and are characterized by an intrinsic uncertainty in the measurements.

Therefore, given the uncertainty values of the measurement typically indicated by the manufacturer of the device, the values are all pre-processed inside a block called the pre-processor of uncertainty 2220. All the outputs of the pre-processor of uncertainty are fully carrying the uncertainty values. The uncertainty values are therefore passed to the orientation block 2230, provided by the 3-axis IMU, and the images taken by the camera resulting in the visual inertial odometry block 2240. Camera images may be used to estimate the velocity of the system as it moves, calculating the displacement between sequential images and their direction and orientation in order to keep the search box area established by the doctor or the sonographer always in the field of view.

Outputs from the visual inertial odometry, 3-axis IMU and orientation are then passed to the filtering system 2280 composed of four main blocks: the odometry velocity measurement block, the orientation roll-pitch-yaw measurement, position measurement and ultrasound measurement. In order to provide 2D/3D images the filtering system uses the technique of particle filtering. Real-time measurements coming from all the synchronized sensors are used to build the layer of the heat grid map at that specific depth given the penetration rate of the laser. In order to predict what the next measurement will be (i.e. the next depth measurement) historical and current vibrational data points are used to predict future vibrational points, providing insights into what the next depth measurement might be.

The representation of the four blocks and therefore the vibrational layer (heat grid map) at a specific depth are therefore visualized in a 3D visualizer monitor 2296. The real-time system related to the architecture shown in FIG. 5, FIG. 7, FIG. 9 or FIG. 11 is equipped with an information logger 2297 able to record every input/output of each sensor in case the user would like to change or tune some of the sensors parameters to carry additional studies post ultrasound exam or perform different simulation and given the movement of all the external sensors and their local relative reference frames with relation to the absolute reference frame provided by the GPS.

By way of example: Q-Switched laser A sends a pulse a time 0 at position 0 so the first wave can penetrate the surface of the skin and arrive at the preferred depth. Q-Switched laser B sends a pulse, for example, 10 nanoseconds later a time 1 at position 1 so the second wave can penetrate the surface of the skin and arrive at the preferred depth. The interval between the two pulses from the two different lasers is when the maximum surface wave vibration is detected from the external interferometer. So in real-time, the system translates (or roto-translates) at extremely low speed within the search area defined by the doctor while it performs the scan alternating the two pulses from the two different lasers. Maximum vibrational surface points are located by the interferometer and then translated into a proper grid map after compensating for rotation and scale. The particle filter algorithm can be used to predict what the next maximum vibrational point can be as it is able to find patterns in historical and current data to extend them into future predictions. In this way real-time is possible.

The additional block 2294 shows that images properly processed may be subjected to an additional matching step where outside stereo images taken using the cameras overlap with the vibrometric images (grid heat map) optically detected at that depth using the interferometer. So any vibrational point outside of the search area can be considered an outlier and, therefore, neglected in real-time.

After patient A finishes an ultrasound session, the doctor has the 2D/3D heat grid map. If patient A comes back a few months later the doctor may upload the previous vibrational map as an input 2290 on the ultrasound measurement block after properly relating the previous reference frame from the initial session with the current reference frame of the new ultrasound session using a rotation matrix 2293 and at the same time perform a new scan. At the end of the ultrasound session, the doctor may compare the two vibrational maps to see if any changes inside the tissue occurred and act accordingly. The main reason of the block 2293 would be that between the two ultrasound sessions, the system in FIG. 5, FIG. 7, FIG. 9 or FIG. 11 could have been subjected to recalibration. Therefore, making sure that the reference frames are oriented the same is important to give the correct interpretation to the heat grid map images.

EXAMPLE 23: Flow chart explaining the post-processing procedure and image reconstruction for two sources Q-Switched pulsed laser working simultaneously and in synchronization, colored camera, black and white camera, interferometer, GPS and 3-axis IMU.

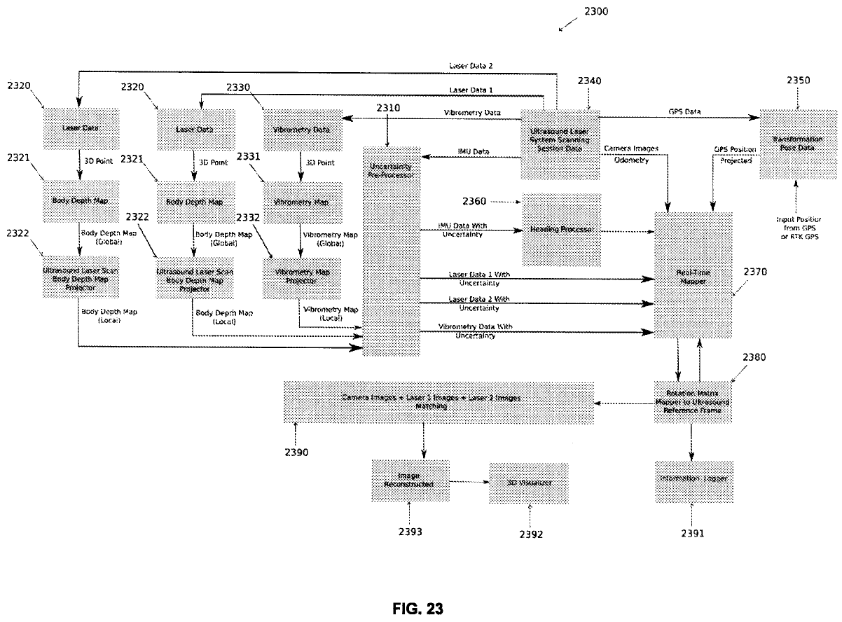

Referring to FIG. 23, a flow chart setting forth the most important steps for post-processing and generating laser images is provided. To start the post-processing loop and generate 2D/3D laser images (i.e. playing back an ultrasound session using a Robotic Platform such as ROS) related to the architecture shown in FIG. 5, FIG. 7, FIG. 9 or FIG. 11, the user needs to start from where all the data from the scanning session is collected, which is the scanning session data 2340. The ultrasound lasers system scanning session data 2340 is the main container of the recorded ultrasound session. Such data are, for example, displacement, position, velocity and acceleration of every sensor and even monitoring the movements of the stepper motors. The presence of the GPS makes sure that there is an absolute reference frame from which all the other local reference frames given from the other sensors relate to while the system translates or roto-translate around the patient.

All necessary data coming from laser sources, external sensors such as color camera, black and white camera, 3-axis IMU, interferometer and a GPS, or an RTK GPS antenna system, 2350 are characterized by an intrinsic uncertainty in the measurements.

Therefore, given the uncertainty values of the measurement typically indicated by the manufacturer of the device, the values are all pre-processed inside a block called the pre-processor of uncertainty 2310. All the outputs of the pre-processor of uncertainty are fully carrying the uncertainty values. In this example vibrational data and laser data extracted are also subjected to uncertainty calculation. The main reason for this variation is due to the fact that between the laser pulses from the two lasers the vibration will be detected on the surface with a certain delay from the interferometer. This delay may cause a small uncertainty value in the measurement of depth. Therefore that delay, or uncertainty value in the calculation, can also be taken in consideration and update the depth measurement accordingly.

Other measurements such as 3-axis IMU data with uncertainty of the other sensors, need further processing as it is required to transform all the necessary orientation, linear and angular velocities into a specific direction. This is even more important in the case of a hand-held device or a head wearable device. The 3-axis IMU data with uncertainty needs to be transformed therefore into heading via a heading processor 2360. After that these 3-axis IMU values can be processed in the real-time mapper system 2370. An additional sensor such as a GPS, or an RTK GPS antenna system, represented by the block 2350 can be implemented to set the absolute reference frame.

In order to make sure that the images are properly reconstructed, a proper transformation via rotation matrix 2380 from Real-Time Mapper to Ultrasound Reference frame may be required as shown in FIG. 23. Displacement of laser images may allow for feature detection and therefore point-to-point registration allowing for a better image formation. In addition and at later steps, outliers may also be neglected for a better performance of the calculation. The additional block 2390 shows that images properly processed may be subjected to an additional matching step where outside stereo images taken using the cameras overlap with the vibrometric images (grid heat map) optically detected at that depth using the interferometer. So any vibrational point outside of the search area can be considered an outlier and, therefore, neglected.

Results of the exam are shown into a 3D visualizer 2392. The post-processing system related to the architecture shown in FIG. 5, FIG. 7, FIG. 9 or FIG. 11 is equipped with an information logger 2391 able to record every input/output of each sensor in case the user would like to change or tune some of the sensors parameters to carry additional studies post ultrasound exam or perform different simulation and experimenting specific scenarios by modifying specific parameters. Therefore, using the Robotic Platform, makes it possible to playback the ultrasound session for that specific patient given the recording of the movement of all the external sensors and their local relative reference frames with relation to the absolute reference frame provided by the GPS.

EXAMPLE 24: Flow chart explaining the post-processing procedure and image reconstruction for a one source Q-Switched pulsed laser, colored camera, black and white camera, interferometer, GPS and 3-axis IMU.

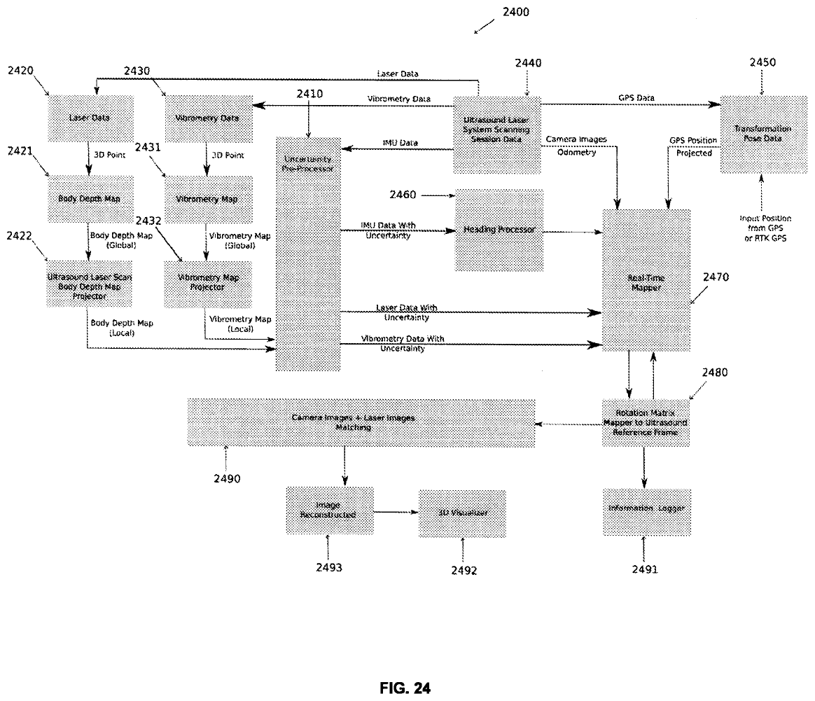

Referring to FIG. 24, a flow chart setting forth the most important steps for post-processing and generating laser images is provided. To start the post-processing loop and generate 2D/3D laser images (i.e. playing back an ultrasound session using a Robotic Platform such as ROS) related to the architecture shown in FIG. 5, FIG. 7, FIG. 9 or FIG. 11, the user needs to start from where all the data from the scanning session is collected, which is the scanning session data 2440. The ultrasound laser system scanning session data 2440 is the main container of the recorded ultrasound session. Such data are, for example, displacement, position, velocity and acceleration of every sensor and even monitoring the movements of the stepper motors. The presence of the GPS makes sure that there is an absolute reference frame from which all the other local reference frames given from the other sensors relate to while the system translates or roto-translate around the patient.

All necessary data coming from laser source, external sensors such as color camera, black and white camera, 3-axis IMU, interferometer and a GPS, or an RTK GPS antenna system, 2450 are characterized by an intrinsic uncertainty in the measurements.

Therefore, given the uncertainty values of the measurement typically indicated by the manufacturer of the device, the values are all pre-processed inside a block called the pre-processor of uncertainty 2410. All the outputs of the pre-processor of uncertainty are fully carrying the uncertainty values. In this example vibrational data and laser data extracted are also subjected to uncertainty calculation. The main reason for this variation is due to the fact that between the laser pulses the vibration will be detected on the surface with a certain delay from the interferometer. This delay may cause a small uncertainty value in the measurement of depth. Therefore that delay, or uncertainty value in the calculation, can also be taken in consideration and update the depth measurement accordingly.

Other measurements such as 3-axis IMU data with uncertainty of the other sensors, need further processing as it is required to transform all the necessary orientation, linear and angular velocities into a specific direction. This is even more important in the case of a hand-held device or a head wearable device. The 3-axis IMU data with uncertainty needs to be transformed therefore into heading via a heading processor 2460. After that these 3-axis IMU values can be processed in the real-time mapper system 2470. An additional sensor such as a GPS, or an RTK GPS antenna system, represented by the block 2450 can be implemented to set the absolute reference frame.

In order to make sure that the images are properly reconstructed, a proper transformation via rotation matrix 2480 from Real-Time Mapper to Ultrasound Reference frame may be required as shown in FIG. 24. Displacement of laser images may allow for feature detection and therefore point-to-point registration allowing for a better image formation. In addition and at later steps, outliers may also be neglected for a better performance of the calculation. The additional block 2490 shows that images properly processed may be subjected to an additional matching step where outside stereo images taken using the cameras overlap with the vibrometric images (grid heat map) optically detected at that depth using the interferometer. So any vibrational point outside of the search area can be considered an outlier and, therefore, neglected.

Results of the exam are shown into a 3D visualizer 2492. The post-processing system related to the architecture shown in FIG. 1 and FIG. 3 is equipped with an information logger 2491 able to record every input/output of each sensor in case the user would like to change or tune some of the sensors parameters to carry additional studies post ultrasound exam or perform different simulation and experimenting specific scenarios by modifying specific parameters. Therefore, using the Robotic Platform, makes it possible to playback the ultrasound session for that specific patient given the recording of the movement of all the external sensors and their local relative reference frames with relation to the absolute reference frame provided by the GPS.

EXAMPLE 25: Flow chart explaining the post-processing procedure and image reconstruction for two sources Q-Switched pulsed laser working simultaneously and in synchronization, colored camera, black and white camera, interferometer, GPS and 3-axis IMU.

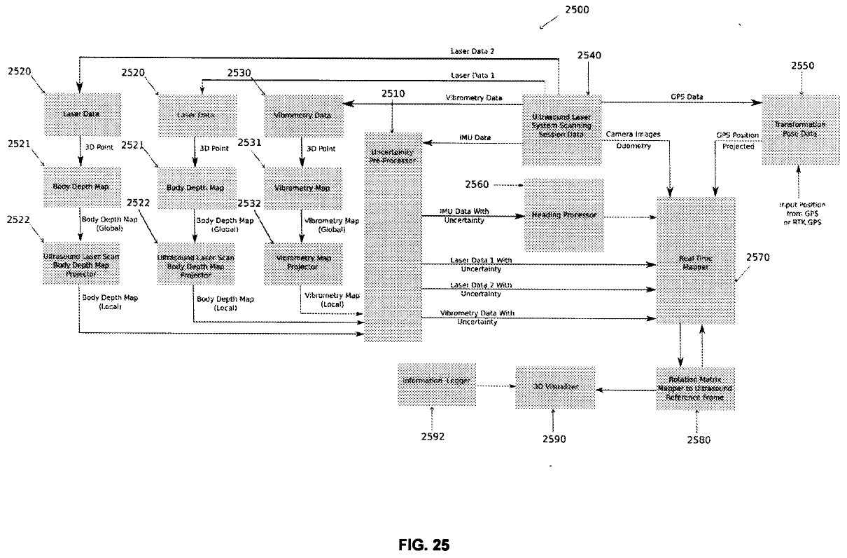

Referring to FIG. 25, a flow chart setting forth the most important steps for post-processing and generating laser images is provided. To start the post-processing loop and generate 2D/3D laser images (i.e. playing back an ultrasound session using a Robotic Platform such as ROS) related to the architecture shown in FIG. 5, FIG. 7, FIG. 9 or FIG. 11, the user needs to start from where all the data from the scanning session is collected, which is the scanning session data 2540. The ultrasound lasers system scanning session data 2540 is the main container of the recorded ultrasound session. Such data are, for example, displacement, position, velocity and acceleration of every sensor and even monitoring the movements of the stepper motors. The presence of the GPS makes sure that there is an absolute reference frame from which all the other local reference frames given from the other sensors relate to while the system translates or roto-translate around the patient.

All necessary data coming from laser sources, external sensors such as color camera, black and white camera, 3-axis IMU, interferometer and a GPS, or an RTK GPS antenna system, 2550 are characterized by an intrinsic uncertainty in the measurements.

Therefore, given the uncertainty values of the measurement typically indicated by the manufacturer of the device, the values are all pre-processed inside a block called the pre-processor of uncertainty 2510. All the outputs of the pre-processor of uncertainty are fully carrying the uncertainty values. In this example vibrational data and laser data extracted are also subjected to uncertainty calculation. The main reason for this variation is due to the fact that between the laser pulses from the two lasers the vibration will be detected on the surface with a certain delay from the interferometer. This delay may cause a small uncertainty value in the measurement of depth. Therefore that delay, or uncertainty value in the calculation, can also be taken in consideration and update the depth measurement accordingly.

Other measurements such as 3-axis IMU data with uncertainty of the other sensors, need further processing as it is required to transform all the necessary orientation, linear and angular velocities into a specific direction. This is even more important in the case of a hand-held device or a head wearable device. The 3-axis IMU data with uncertainty needs to be transformed therefore into heading via a heading processor 2560. After that these 3-axis IMU values can be processed in the real-time mapper system 2570. An additional sensor such as a GPS, or an RTK GPS antenna system, represented by the block 2550 can be implemented to set the absolute reference frame.

In order to make sure that the images are properly reconstructed, a proper transformation via rotation matrix 2580 from Real-Time Mapper to Ultrasound Reference frame may be required as shown in FIG. 25. Displacement of laser images may allow for feature detection and therefore point-to-point registration allowing for a better image formation. In addition and at later steps, outliers may also be neglected for a better performance of the calculation.

Results of the exam are shown into a 3D visualizer 2590. The post-processing system related to the architecture shown in FIG. 5, FIG. 7, FIG. 9 or FIG. 11 is equipped with an information logger 2592 able to record every input/output of each sensor in case the user would like to change or tune some of the sensors parameters to carry additional studies post ultrasound exam or perform different simulation and experimenting specific scenarios by modifying specific parameters. Therefore, using the Robotic Platform, makes it possible to playback the ultrasound session for that specific patient given the recording of the movement of all the external sensors and their local relative reference frames with relation to the absolute reference frame provided by the GPS.

EXAMPLE 26: Flow chart explaining the post-processing procedure and image reconstruction for a one source Q-Switched pulsed laser, colored camera, black and white camera, interferometer, GPS and 3-axis IMU.

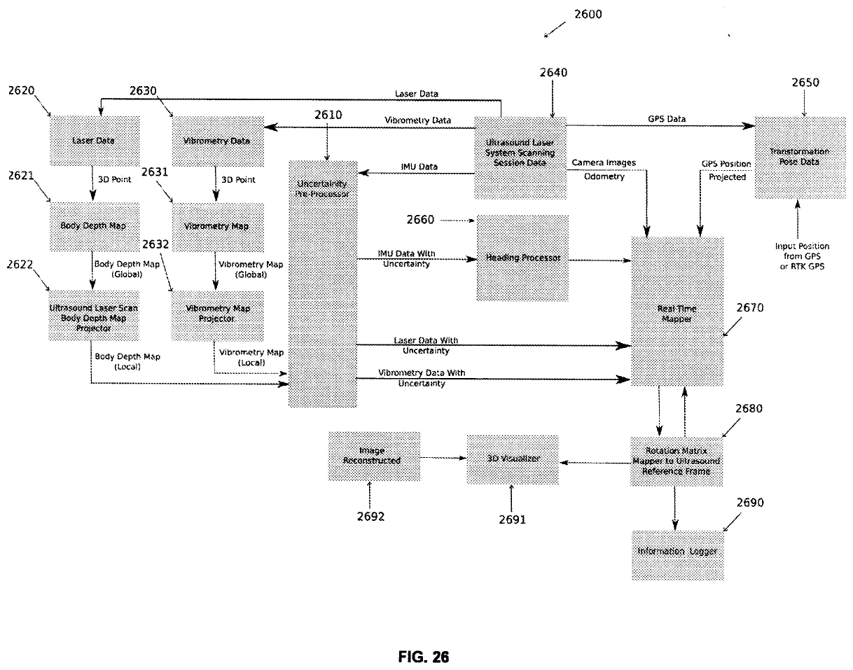

Referring to FIG. 26, a flow chart setting forth the most important steps for post-processing and generating laser images is provided. To start the post-processing loop and generate 2D/3D laser images (i.e. playing back an ultrasound session using a Robotic Platform such as ROS) related to the architecture shown in FIG. 1 and FIG. 3 the user needs to start from where all the data from the scanning session is collected, which is the scanning session data 2640. The ultrasound laser system scanning session data 2640 is the main container of the recorded ultrasound session. Such data are, for example, displacement, position, velocity and acceleration of every sensor and even monitoring the movements of the stepper motors. The presence of the GPS makes sure that there is an absolute reference frame from which all the other local reference frames given from the other sensors relate to while the system translates or roto-translate around the patient.

All necessary data coming from laser source, external sensors such as color camera, black and white camera, 3-axis IMU, interferometer and a GPS, or an RTK GPS antenna system, 2650 are characterized by an intrinsic uncertainty in the measurements.

Therefore, given the uncertainty values of the measurement typically indicated by the manufacturer of the device, the values are all pre-processed inside a block called the pre-processor of uncertainty 2610. All the outputs of the pre-processor of uncertainty are fully carrying the uncertainty values. In this example vibrational data and laser data extracted are also subjected to uncertainty calculation. The main reason for this variation is due to the fact that between the laser pulses the vibration will be detected on the surface with a certain delay from the interferometer. This delay may cause a small uncertainty value in the measurement of depth. Therefore that delay, or uncertainty value in the calculation, can also be taken in consideration and update the depth measurement accordingly.

Other measurements such as 3-axis IMU data with uncertainty of the other sensors, need further processing as it is required to transform all the necessary orientation, linear and angular velocities into a specific direction. This is even more important in the case of a hand-held device or a head wearable device. The 3-axis IMU data with uncertainty needs to be transformed therefore into heading via a heading processor 2660. After that these 3-axis IMU values can be processed in the real-time mapper system 2670. An additional sensor such as a GPS, or an RTK GPS antenna system, represented by the block 2650 can be implemented to set the absolute reference frame.

In order to make sure that the images are properly reconstructed, a proper transformation via rotation matrix 2680 from Real-Time Mapper to Ultrasound Reference frame may be required as shown in FIG. 26. Displacement of laser images may allow for feature detection and therefore point-to-point registration allowing for a better image formation. In addition and at later steps, outliers may also be neglected for a better performance of the calculation.

Results of the exam are shown into a 3D visualizer 2691. The post-processing system related to the architecture shown in FIG. 1 and FIG. 3 is equipped with an information logger 2692 able to record every input/output of each sensor in case the user would like to change or tune some of the sensors parameters to carry additional studies post ultrasound exam or perform different simulation and experimenting specific scenarios by modifying specific parameters. Therefore, using the Robotic Platform, makes it possible to playback the ultrasound session for that specific patient given the recording of the movement of all the external sensors and their local relative reference frames with relation to the absolute reference frame provided by the GPS.

It would be appreciated by those skilled in the art that various changes and modifications can be made to the illustrated embodiments without departing from the spirit of the present invention. All such modifications and changes are intended to be within the scope of the present invention except as limited by the scope of the appended exemplary claims.

While there is shown and described herein certain specific structure embodying the invention, it will be manifest to those skilled in the art that various modifications and rearrangements of the parts may be made without departing from the spirit and scope of the underlying inventive concept and that the same is not limited to the particular forms herein shown and described except insofar as indicated by the scope of the appended claims.

### List of Embodiments and Examples

Specific systems and methods of obtaining laser ultrasound images completely contactless have been described. The detailed description in this specification is illustrative and not restrictive or exhaustive. The detailed description is not intended to limit the disclosure to the precise form disclosed. Other equivalents and modifications besides those already described are possible without departing from the inventive concepts described in this specification, as those skilled in the art will recognize. When the specification or claims recite methods, steps or functions in order, alternative embodiments may perform the tasks in a different order or substantially concurrently. The inventive subject matter is not to be restricted except in the spirit of the disclosure.

When interpreting the disclosure, all terms should be interpreted in the broadest possible manner consistent with the context. Unless otherwise defined, all technical and scientific terms used in this specification have the same meaning as commonly understood by one of ordinary skills in the art to which this invention belongs. This invention is not limited to the particular methodology, systems, protocols, and the like described in this specification and, as such, can vary in practice. The terminology used in this specification is not intended to limit the scope of the invention, which is defined solely by the claims.

What is claimed is:

1. A system comprising of:
a) two photoacoustic laser excitation sources working simultaneously and in synchronization to direct ultrasonic waves into a desired area of tissue via a lens apparatus distributing acoustic energy into the tissue at the speed of sound;
b) two photoacoustic laser excitation sources working simultaneously, in conjunction with one another, and in synchronization with minimal wavelength displacement in order to form laser stereometry images;
c) a receiver device configured to optically detect vibrations at the surface of tissue coming from the two photoacoustic laser excitation sources;
d) a data acquisition system configured to process data originating from the backscattering of ultrasonic waveform sources on the surface of the tissue;
e) a processor system configured to detect ultrasonic waveform sources to assess an internal structure of organs and tissues;
f) a robotic platform configured to include the two laser sources working simultaneously and in synchronization, the interferometric receiving system, the GPS, the color camera, the black and white camera and the stepping motors used for translation and roto-translation;
g) a 3-axis IMUs mounted on each external sensor;
h) and two focused compensation lenses for directing the photoacoustic excitation laser sources into the tissue or the area of interest.

2. A system comprising of:
a) two photoacoustic laser excitation sources working simultaneously and in synchronization to direct ultrasonic waves into a desired area of tissue at the speed of sound;
b) two photoacoustic laser excitation sources working simultaneously, in conjunction with one another, and in synchronization with minimal wavelength displacement in order to form laser stereometry images;
c) a receiver device configured to optically detect vibrations at surface of tissue following the photoacoustic excitation;
d) a data acquisition system configured to process data originating from the backscattering of ultrasonic waveform sources on the surface of the tissue;
e) a processor system configured to detect ultrasonic waveform sources to assess an internal structure of organs and tissues;
f) a robotic platform configured to include the two laser sources working simultaneously and in synchronization, the interferometric receiving system, the GPS, the color camera, the black and white camera and the stepping motors used for translation and roto-translation;
g) a 3-axis IMUs mounted on each external sensor;
h) and a roto-translation apparatus composed of two laser sources working simultaneously and in synchronization, the interferometric receiving system, the GPS, the color camera, the black and white camera and the stepping motors with 3-axis IMU mounted on each of these sensors.

3. A method for generating real-time laser ultrasound 2D-3D images of a subject comprised of:
a) drawing specific points on the skin of the patient with a highlighter and connect them allowing the external color camera and black and white camera to feature-detect those points and establish a specific search area box;
b) two photoacoustics excitation sources working simultaneously, in conjunction with one another and in synchronization with minimal wavelength displacement range emitting ultrasonic waves into a tissue within the search area box determined by the doctor;
c) using an optical interferometer to detect the maximum vibrational points on the surface of the tissue within the search area box determined by the doctor by assigning xyz coordinates to every point;
d) combining all the vibrational coordinate points together to create a heat grid map at a specific depth according to the penetration power of the two photoacoustic excitation sources;
e) repeating process at several depths so that the sonographer can generate different layers at different depths of the same search area box;
f) interpolating the different layers to form a completely contactless 3D image;
g) applying particle filtering techniques to predict a 2D/3D image at another specific depth;
h) visualizing the 2D-3D reconstructed images into a proper visualization processor system;
i) and saving sensor data positions, orientation, displacement, velocities and vibrational points to a data logger.
